(19) United States
(12) Patent Application Publication
(10) Pub. No.: US 2003/0060487 A1
(43) Pub. Date: Mar. 27, 2003

(54) Treatment of neurodegenerative disease

(76) Inventors: R. Shoshana Bamdad, Newton, MA (US); Cynthia C. Bamdad, Newton, MA (US)

Correspondence Address: WOLF GREENFIELD & SACKS, PC, FEDERAL RESERVE PLAZA, 600 ATLANTIC AVENUE, BOSTON, MA, 02210-2211, US

(21) Appl. No.: 09/835,099

(22) Filed: Apr. 12, 2001

Related U.S. Application Data
(60) Provisional application No. 60/196,497, filed on Apr. 12, 2000.
(60) Provisional application No. 60/214,221, filed on Jun. 23, 2000.
(60) Provisional application No. 60/248,890, filed on Nov. 15, 2000.

(51) Int. Cl. A61K 31/454; A61K 31/445; A61K 31/40; A61K 31/44

(52) U.S. Cl. 514/326; 514/317; 514/340; 514/341; 514/408

(57) ABSTRACT

In some aspects, the invention relates to treatments for peptide aggregation associated with disease states such as neurodegenerative disease, particularly physiology associated with Alzheimer's Disease, and non-neurodegenerative disease aggregation. Other aspects of the invention provide a variety of novel assays for screening candidate drugs. Yet other aspects of the present invention provide a series of compositions useful for treatment of peptide aggregation associated with disease states such as neurodegenerative disease, as determined from these assays. These compositions can be packaged in kits. Other aspects of the invention relate to the use of these compositions for the treatment and/or prevention of patients susceptible to or exhibiting symptoms characteristic of fibril formation or aberrant protein aggregation.

### RELATED APPLICATIONS

This non-provisional application claims the benefit under Title 35, U.S.C. §19(e) of co-pending U.S. provisional application Nos. 60/196,497 filed Apr. 12, 2000, 60/214,221 filed Jun. 23, 2000, and 60/248,890 filed Nov. 15, 2000, all incorporated herein by reference.

### FIELD OF THE INVENTION

The invention relates to treatments for peptide aggregation associated with disease states such as neurodegenerative disease, particularly physiology associated with Alzheimer's Disease, and non-neurodegenerative disease aggregation.

### BACKGROUND OF THE INVENTION

A variety of diseases are characterized by abnormal and toxic aggregation of biological molecules such as proteins and peptides, sometimes called protein condensation, tangle formation, fibril formation, plaque formation, etc. An important class of disease associated with abnormal protein aggregation is neurodegenerative disease. Neurodegenerative diseases to which the invention is related include, without limitation (with their associated proteins involved in aggregation), Familial British Dimentia (ABRI), Parkinson's Disease (α-synnuclein), Alzheimer's Disease (Aβ peptide), Finnish-type Familial Amyloidoses (Gelsoin), Huntington's Disease (Huntington), AD, Frontotemporal Dementia (Tau), Senile Systemic Amyloidosis (Transthyretin), Familial Amyloid Polyneuropathy (TTR), and Transmissible Spongiform Encephalopathie (PrP). Many neurodegenerative diseases have now been shown to be linked to and/or caused by plaque associated with aggregate formation in the brain that occurs as a result of aberrant aggregation of neuro peptides. Neurodegenerative diseases including Alzheimer's, Parkinson's, Gertsmann-Strausseler-Scheinker Syndrome, Fatal Familial Insomnia, Huntington's Chorea, Kuru, and Familial amyloid polyneuropathy and transmissible spongiform encephalopathies such as Creutzfeldt Jakob, Scrapie, and Bovine Spongiform Encephalopathy (BSE, Mad Cow), are characterized by ordered protein aggregates that form in the brain. Although the proteins that make up these aggregates share no sequence homology, or even conserved motif, the aggregates themselves share certain morphological features. See Lansbury, Proc. Natl. Acad. Sci. USA, 96:3342 (1999). In these diseases, peptides related to the pathogenic state, which are normally soluble, undergo a conversion of their 3-dimensional structure via a mutation of the native peptide or a physical association with altered peptides, to an insoluble, ordered polymerized state that is characteristic of the abnormal protein deposits that are found in the brain in neurodegenerative diseases. This ordered polymerizing or aggregation may result from seeding with indogenous or exogenous agents or peptides.

For example, in Alzheimer's disease (AD), these aggregates are made up of β-amyloid protein that has undergone a conformational change, from soluble monomers, to insoluble, β-sheet oligomers. The concentration of these fibrils in the brain has been correlated to the progression of clinical disease. The growth profile of the characteristic fibrils is extremely non-linear, which could explain why its victims can appear asymptomatic for years, then suddenly undergo a rapid degeneration into dementia. Fibril formation in vitro is peptide concentration dependent. Short synthetic peptides, derived from the β-amyloid (Aβ) protein, can be made to form fibrils in vitro to mimic fibril formation that is characteristic of Alzheimer's disease. Aβ 1-42 (with an extended hydrophobic C-terminus) has been shown to form fibrils at a faster rate than Aβ 1-40. See J. Jarrett J et al, “The carboxy terminus of the β-amyloid protein is critical for the seeding of amyloid formation: implications for the pathogenesis of Alzheimer's disease,” Biochemistry 32: 4693-4697 (1993). While Aβ 1-40 can form fibrils or aggregates on its own, solutions containing Aβ 1-40 undergo accelerated fibril formation if they are “mixed” with the less soluble 1-42 peptide or are “seeded” with pre-formed peptide fibrils. Although Aβ 1-40 is the predominant protein in neuritic plaque, these studies indicate that the rate of fibril formation may depend on the ratio of the concentration of 1-42 to 1-40. Consistent with these findings, all forms of early onset AD involve higher expression levels of the 1-42 peptide.

Atomic force microscopy (AFM) studies of in vitro fibril formation using the two most prevalent variants of Aβ, 1-42 and 1-40, have identified a meta-stable intermediate, termed the protofibril that occurs before fibril formation. See J. Harper et al, “Observation of metastable Aβ protofibrils by atomic force microscopy,” Chem. and Biol 4: 119-125 (1997). The existence of these precursors to fibrils may explain the diffuse amyloid deposition observed in the brains of people pre-disposed to AD. The existence of a protofibril that is a toxic intermediate would explain several observed inconsistencies in vitro and in vivo that argued both for and against the Aβ fibril being the pathogenic agent of AD. Existing assays to test for neurodegenerative disease fibrils or fibril-forming species, or to screen for drugs suitable for treatment of neurodegenerative disease are Congo red and Thioflavin-T assays (Methods in Enzymology, Academic Press, 1999, Vol. 309, pgs. 274-287; 304-305) which typically cannot detect small aggregates or protofibrils. Specifically, they cannot detect aggregate or fibril-forming species at a concentration below about 100 μM. This is inadequate for detecting early stage disease, and for screening drugs suitable for use at early stage disease.

Drug candidates that act at a pre-symptomatic stage of the disease (when only small fibrils or aggregates are present) would have a greater efficiency in inhibiting plaque/fibril formation and preventing symptomatic disease. In order to do this, small fibril aggregates need to be efficiently detected. However, these aggregates are too small to be detected by nearly every detection method. Although they can be detected by AFM, this technique does not lend itself to clinical diagnostics or drug screening protocols. Therefore, at present, it has not been possible to screen for drugs that would act on the smaller fibrillar species. Additionally, screening for drugs to inhibit fibril formation at any stage has been severely limited, thus severely limiting the number of drugs available for treatment.

The rate of aggregate and fibril formation is an extremely non-linear function of the concentration of converted or misfolded peptide, such as mutant neuro peptide or converted prion peptides, which are aggregate-forming or fibril-forming species. Once the concentration of the aberrant species reaches a critical concentration, the reaction rate proceeds too quickly to be affected by drug treatment. Therefore, for a drug to be effective at inhibiting plaque formation, thus making it a preventative therapeutic rather than a palliative one, it would necessarily have to act at an early stage. Current state of the art technology is not capable of detecting small aggregates or fibrils in a manner that is compatible with parallel drug screening methods or non-invasive diagnosis. This means that: 1) drugs to treat the early disease state cannot be readily identified; 2) pre-symptomatic patients cannot be identified; and 3) the effectiveness of potential drug candidates that exist, or will be identified in the future, cannot be accurately assessed.

Another drawback of existing technologies, for example, Congo Red and Thioflavin-T is that they require mechanical intervention during the process. That is, the process requires transfer of fluid from one container to another, and the like which disturbs the assay in a non-reproducible way accelerating fibril formation. Additionally, addition of the Congo Red and Thioflavin components quenches the reaction and stops the aggregation process, thus several time points cannot be taken of a single assay in solution.

One complication with the diagnosis of neurodegenerative disease is the fact that species capable of forming aggregates or fibrils characteristic of the disease may be present in extremely low concentration, yet at a concentration which, if detectable, could signify onset of the disease.

There are also many non-neurodegenerative diseases that involve aberrant protein aggregation. These diseases include but are not limited to the following: In multiple myeloma, antibody light chain aggregates to cause toxic effects. Waldenstroms Macroglobulinemia is a disease characterized by antibody heavy chain aggregation. A class of proteins, called cryoglobulins, precipitates at low temperatures, causing blockage of affected blood vessels. Disseminated intravascular coagulation (DIC) is a major cause of morbidity and mortality in people with severe systemic infections or autoimmune diseases, presumably because it depletes fibrin, resulting in an inhibition of blood clotting. Glanzmann's thrombasthenia is a bleeding disorder, characterized by abnormal platelet aggregation due to a defect in the integrin, alpha Iib beta 3¹. Abnormal fibronectin aggregation is characteristic of genetic human fibronectin-deposit glomerular disease². Sickle cell anemia is caused by mutant hemoglobin that aggregates rather than forming tetramers³. Abnormal protein aggregation has also been implicated as a toxic factor in stroke. ¹MorelKopp M C, Kaplan C, proulle V, Jallu V, Melchlor C, Peyruchaud O, Aurousseau M H, and Kieffer N, “A three amino acid deletion “, Blood, 90 (2) 669-677 1997. ²Zhang Z J, Kundu G C, Yuan C J, Ward J M, Lee E J, DeMayo F, Westphal H, and Mukherjee A B, “Severe fibronectin-deposit renal glomerular disease in mice lacking uteroglobin”, Science, 1997, 276 (5317) 1408-1412. ³Li X F, Himanen J P, deLlano J J M, Padovan J C, Chait B T, and Manning J M, “Mutational analysis of sickle haemoglobin (Hb) gelation”, Biotechnology and Applied Biochemistry, 1999, 29, 165-184.

Of particular importance is abnormal protein aggregation involved in Type II Diabetes. Human islet amyloid polypeptide (hIAPP) is the major component of plaques found in the pancreatic islets of Langerhans in people who have type II diabetes mellitus⁴. Amino acids 20-29 have been shown to be responsible for this aggregation. The aggregation of hIAPP proceeds non-linearly as a function of time and can be accelerated by “seeding” the reaction with pre-formed aggregates, analogous to the case of beta-amyloid aggregation characteristic of Alzheimer's disease. However, unlike Alzheimer's disease, aggregation of hIAPP is independent of peptide concentration. An off-aggregation peptide micelle model has been suggested to explain this. ⁴Rhoades E., Agarwal J., Gafni A., “Aggregation of an amyloidogenic fragment of human islet amyloid polypeptide”, Biochemica Et Biophysica Acta-Protein Structure and Molecular Enzymology, Vol. 1476 (2) 230-238, February, 2000.

While advances and discoveries have been made in connection with diseases characterized by abnormal protein aggregation, a need exists for new drugs for treatment of such diseases.

### SUMMARY OF THE INVENTION

The present invention involves, in one aspect, methods for treating patients susceptible or exhibiting symptoms of a neurodegenerative disease such as Alzheimer's Disease. In another aspect, the invention relates to the discovery of a variety of compositions (e.g. drugs) useful for inhibition of formation of aggregates characteristic of neurodegenerative disease. These compositions can be provided in a kit including instructions for use of the composition for treatment of diseases. Assays can be performed to screen for and identify such compositions and also for identifying which compositions are effective at various stages of the disease process. For example, International Pat. Apl. Ser. No: PCT/US00/01997, filed Jan. 25, 2000, entitled, “Rapid and Sensitive Detection of Aberrant Protein Aggregation in Neurodegenerative Diseases”, by Bamdad and Bamdad, the disclosure of which is incorporated herein by reference, describes several assays which are useful for determining composition suitable for treatment of neurodegenerative disease, or patients susceptible to neurodegenerative disease. Other assays are described in the Examples herein.

Also included is a combinatorial approach in which structural features identified as characteristic of compositions effective for treatment at various disease stages are used as the basis for combinatorial synthesis of a wide variety of structural analogs for identification of a wide variety of compositions also useful for treatment.

In another method of the invention, a single patient is treated with at least two different compositions, one composition being more effective in inhibiting neurodegenerative disease aggregate formation at a first stage of formation, and the second composition being more effective at inhibiting aggregate formation at a second stage of aggregate formation. For example, a first disease stage may be optimally treated by providing a composition that inhibits aggregate formation, while a second disease stage may optimally be treated by administering a composition that inhibits aggregate formation and, in combination, administering a composition that inhibits further aggregate formation once initial aggregate formation has occurred. In accordance with this method, a patient can be treated with a single composition identified as effective at a particular stage of aggregate or plaque formation, or a single patient may be treated with a plurality of compositions that target a variety of stages. These stages can span formation from beginning to end, including initial targeting of monomeric species for prevention of aggregate or fibril formation through various intermediate species and including mature plaque formation.

In another aspect of the invention a series of compositions of matter have been identified as suitable for treatment of a patient susceptible to or exhibiting symptoms of a neurodegenerative disease such as Alzheimer's Disease, by administering to a patient a therapeutically effective amount of a composition able to cross the blood/brain barrier and having structures as defined below.

Another aspect of the invention also provides a method involving promoting the prevention or treatment of a neurological disease via administration of a composition having a structure as defined below, and derivatives and enantiomers thereof.

In another aspect the invention provides a kit including a composition having a structure among those described below, and instructions for use of the composition for treatment of neurological disease.

Several method are disclosed herein of administering a subject with a compound for prevention or treatment of a particular condition. It is to be understood that in each such aspect of the invention, the invention specifically includes, also, use of the compound for the manufacture of a medicament for the treatment or prevention of that particular condition.

These and other aspects of the invention will be described in further detail in connection with the detailed description of the invention.

### BRIEF DESCRIPTION OF THE FIGURES

FIG. 1 is a color photocopy of a CCD photograph of one exemplary assay plate used in some embodiments of the invention, and representative photocopies of optical micrographs of various wells of the plate;

FIG. 2 is a graph showing drug activity as a function of time in a neurodegenerative disease assay, correlating drug activity to aggregate size;

FIG. 3 shows structures A-I of drugs identified by a whole cell assay that effect APP production;

FIG. 4 shows structures J-N of drugs identified by a whole cell assay that effect APP production;

FIG. 5 shows structures of drugs which can be used in some embodiments of the invention;

FIG. 6 shows structures of drugs which can be used in some embodiments of the invention;

FIG. 7 shows structures of drugs which can be used in some embodiments of the invention;

FIG. 8 shows structures of drugs which can be used in some embodiments of the invention;

FIG. 9 shows structures of drugs which can be used in some embodiments of the invention; and

FIG. 10 shows structures of drugs which can be used in some embodiments of the invention.

### DETAILED DESCRIPTION

International patent application serial number PCT/US00/01997, filed Jan. 25, 2000 by Bamdad et al., entitled “Rapid and Sensitive Detection of Aberrant Protein Aggregation in Neurodegenerative Diseases” (International patent publication WO 00/43791, published Jul. 27, 2000), International patent application serial number PCT/US00/01504, filed Jan. 21, 2000 by Bamdad, et al, entitled “Interaction of Colloid-Immobilized Species with Species on Non-Colloidal Structures” (International patent publication WO 00/34783, published Jul. 27, 2000), commonly-owned, copending U.S. patent application filed on even date herewith by Bamdad et al., entitled “Rapid and Sensitive Detection of Protein Aggregation”, and commonly-owned, copending U.S. patent application filed on even date herewith by Bamdad et al., entitled “Interaction of Colloid-Immobilized Species with Species on Non-Colloidal Structures” all are incorporated herein by reference.

In one aspect, the present invention provides a series of compositions useful for treatment of diseases.

Several tests can be used to identify compounds which are suitable for use in the treatment of neurodegenerative disease. For example, International patent publication WO 00/43791, referenced above, describes several assays which are useful for determining composition suitable for treatment of neurodegenerative disease, or patients susceptible to neurodegenerative disease. The assay can be generalized and/or adapted for identification of essentially any composition useful for treatment of neurodegenerative disease according to some embodiments of the invention, and/or identification of compositions particularly suitable for treatment of specific stages of neurodegenerative plaque formation or its precursors. “Neurodegenerative disease” as used herein is distinguished from disease-associated dementia from mental illness, schizophrenia, or the like.

Another aspect of the present invention also provides a series of compositions useful for treatment of neurological disease, including these compositions packaged in kits including instructions for use of the composition for the treatment of neurological disease. The kits can further include a description of activity of the composition in treating the pathology, as opposed to the symptoms, of neurological disease. That is, the kit can include a description of use of the composition for inhibition of aggregate, as discussed herein. The kit also can include instructions for use of a combination of two or more compositions of some embodiments of the invention. Instructions also may be provided for administering the drug orally, intravenously, or directly into the cerebro-spinal fluid via a spinal drip, pump, or implantable delivery device. These and other embodiments of the invention can also involve promotion of the treatment of neurodegenerative disease according to any of the techniques and compositions and composition combinations described herein.

As mentioned, some embodiments of the invention provide a series of compositions useful for treatment of patients susceptible to or exhibiting symptoms characteristic of neurodegenerative disease. In one set of embodiments, such patients can be treated with these compositions even though the patients exhibit indication for treatment of one of the compositions of the invention for a condition different from neurodegenerative disease, including conditions that can be unrelated to neurodegenerative disease or conditions that can accompany neurodegenerative disease. That is, if a composition of the invention is known for treatment of a different condition, some embodiments of the present invention also involve use of that composition for treatment of neurodegenerative disease where indicated. These and other embodiments of the invention can include such treatment where the dosage, delivery technique or vehicle, combination with other pharmaceutical compositions or lack of combination with other pharmaceutical compositions, rate of administration, timing of administration, or other factor differs from the use of the composition for treatment of the condition different from neurodegenerative disease. In another set of embodiments, treatment of neurodegenerative disease with compositions of the invention may occur under conditions that are similar to or overlap the use of compositions of the invention for treatment of a different condition, but the compositions of the invention are promoted for treatment of neurodegenerative disease or includes instructions for treatment of neurodegenerative disease as mentioned above. As used herein, “promoted” includes all methods of doing business including methods of education, hospital and other clinical instruction, pharmaceutical industry activity including pharmaceutical sales, and any advertising or other promotional activity including written, oral, and electronic communication of any form, associated with compositions of the invention in connection with treatment of neurological disease. “Instructions” can and often do define a component of promotion, and typically involve written instructions on or associated with packaging of compositions of the invention. Instructions also can include any oral or electronic instructions provided in any manner. The “kit” typically, and preferably defines a package including both any one or a combination of the compositions of the invention and the instructions, but can also include the composition of the invention and instructions of any form that are provided in connection with the composition in a manner such that a clinical professional will clearly recognize that the instructions are to be associated with the specific composition.

Subjects for whom the treatment methods of the invention (with specific compositions directed toward neurodegenerative disease) are not intended are those who are diagnosed with a condition which may already call for treatment with the specific composition. Accordingly, one aspect of the invention involves treatment of Alzheimer's with a specific composition disclosed herein for that purpose, not in combination with another agent where the other agent has been taught previously for use in treatment of Alzheimer's itself. Another embodiment involves treatment of Alzheimer's with this specific composition alone, not in combination with any other active agent. Another embodiment involves treatment of Alzheimer's with this specific composition where the use of the composition in the treatment is specifically instructed (through, e.g. written instructions that can accompany the composition) for the treatment of Alzheimer's. In a preferred embodiment of this aspect, the invention involves treatment of Alzheimer's with the specific composition where the use of the composition in the treatment is specifically instructed to reduce, prevent, minimize, or reverse, neurodegenerative aggregate or fibril formation.

In yet another set of embodiments, the invention is particularly directed to a patient population never before treated with drugs useful according to certain methods of the invention, including patients who are not suffering from or indicating susceptibility to neurodegenerative disease such as Alzheimer's Disease. In other words, the treatment preferably is directed to patient populations that otherwise are free of symptoms that call for treatment with any of the drugs useful according to the invention.

As used herein, “a human patient susceptible to Alzheimer's Disease” includes a patient showing genetic mutation or other genetic indication of susceptibility to the disease; a patient showing family history of the disease; a patient showing biochemical indication of susceptibility to the disease including indication using an assay of some embodiments of the invention or an assay as described in International Patent Publication No. WO 00/43791, referenced above; a patient of a minimum age such as at least about 50, 55, or 60 years of age; a patient showing elevated levels of Tau protein in urine or serum, elevated levels of β-amyloid 1-42 in the blood, or the like. “A human patient exhibiting symptoms of Alzheimer's Disease”, means a patient exhibiting memory loss, disorientation, central nervous system neuronal damage, or other symptoms of dementia as caused by aggregate formation, as would be recognized by those of ordinary skill in the art.

Compositions of the invention are given in dosages, generally, given at the maximum amount while avoiding detrimental side effects.

The formulations of the invention are administered in effective amounts, alone or in a cocktail with one or more of the foregoing compounds. An effective amount is one sufficient to inhibit neurodegenerative disease aggregate formation, either at a beginning stage by inhibiting formation of the aggregates from peptides, or at a later stage where aggregates can be formed in part from existing aggregates. “Aggregate” as used herein refers to any collection of homologous molecules as well as mixtures of molecules, typically proteinaceous molecules or the like associated with the onset or susceptibility to a neurodegenerative disease. “Aggregates”, as used herein, encompasses fibrils. All fibrils are aggregates but not all aggregates are fibrils entities, and vice versa.

“Aggregate-forming species”, as used herein, means biological species associated with disease or non-disease processes involving aggregates, as defined above, having sufficient binding capacity to bind to other molecules associated with the disease or non-disease process (including like molecules), to form fibrils or aggregates characteristic of the process. In some disease processes such as neurodegenerative disease, such aggregate-forming species typically are characterized by a change in molecule conformation, relative to sequence-homologous, healthy, counterparts, allowing them to bind more readily to like or similar molecules. In some cases, such as in neurodegenerative disease, type-II diabetes, and myeloma, such aggregate-forming species have the capability to convert binding species from non-aggregate-forming conformation into aggregate-forming conformation. Protofibrils formed from neurodegenerative disease aggregate-forming species have been reported to be a precursor of, or an early from of, neurodegenerative disease associated aggregates. Protofibrils are included in the definition of aggregate-forming species as used herein. Similar to the term “aggregates”, those of ordinary skill in the art understand the meaning of the term “aggregate forming species” as it is applied to neurodegenerative disease aggregate-forming species, non-neurodegenerative disease aggregate-forming species, and non-disease aggregate-forming species.

One of skill in the art can determine what an effective amount of a composition is by screening the ability of the composition to reduce, minimize, reverse, or eliminate aggregate formation in any of the assays described herein. Effective amounts will depend, of course, on the severity of the condition being treated; individual patient parameters including age, physical condition, size and weight; concurrent treatment; frequency of treatment; and the mode of administration. These factors are well known to those of ordinary skill in the art and can be addressed with no more than routine experimentation. It is preferred generally that a maximum dose be used, that is, the highest safe dose according to sound medical judgment.

In one set of embodiments compositions of the invention are administered to patients free of indication for treatment for central nervous system neuronal damage, and/or patients free of symptoms of dementia. In this set of embodiments compositions may have a early-stage, and/or preventative effect. In another set of embodiment compositions of the invention can be administered to those who show indication for treatment for central nervous system neuronal damage and/or show symptoms of dementia as caused by aggregate formation. Compositions of the invention can be useful in preventing the progression of disease even when disease has reached such a stage.

In one embodiment, the method comprises treating the patient with a dose of normal DNA or RNA bases. It is known that certain compositions can be toxic, and thus destroy normal DNA or RNA bases already present in the patient. The addition of normal DNA or RNA bases not only replenishes the supply but allows treating the patient with a higher dosage of the composition to counteract the aggregate forming process.

Another aspect of the invention provides a method of treatment involving drawing a sample from a patient and monitoring the sample over at least two points in time. The term “sample” refers to any cell, tissue, or fluid. For example, a fluid sample is taken from a human suspected of having a disease associated with aggregate formation. Typical samples taken from humans or other animals include cells, blood, urine, ocular fluid, saliva, cerebro-spinal fluid, fluid or other samples from tonsils, lymph nodes, needle biopsies, etc.

In one embodiment, “monitoring” involves subjecting the sample to an assay as described herein or in International Pat. Apl. Publication. No. WO 00/43791, referenced above, followed by detecting the results of the assay. The assay can screen for the presence of aggregates, fibrils, and proto-fibrils (or aggregate-forming species) which can subsequently be used to screen for aggregation-associated diseases. These assays can determine whether particle aggregation occurs via addition of a first and second article each having a surface to the sample (a variety of articles are contemplated) and also addition of a plurality of binding species, at least some of which are immobilized relative to or adapted to be immobilized relative to the surface of the first article, and at least some of which are immobilized relative to or adapted to be immobilized relative to the surface of a second article. The binding species are capable of binding aggregate-forming species. As will become apparent from the detailed description below, a variety of combinations of articles can be used in this embodiment. Each article can be a fluid-suspendable, isolatable article, each being a colloid particle or one being a colloid particle and the other being a magnetic bead or the like, or one article can be a surface of a larger article such as an electrode, a surface plasmon resonance (SPR) chip, or other macroscopic article, and the other article can be a fluid-suspendable particle as described above.

“Detecting” involves any method capable of detecting localization of aggregate or fibril formation, typically in concentrated areas surrounding cells, where a higher level of aggregate or fibril-forming species typically exists. Localization of aggregate or fibril-forming species can be seen as areas of increased peptide or colloid reticulum or as areas where the solution has cleared adjacent areas of increased peptide/colloid reticulum due to drawing of the aggregate or fibril-forming species into the areas of reticulum. Examples of monitoring include visualization with the unaided human eye, e.g. a color change, or change in turbidity of a solution, or visualization via microscopy. Spectrophotometry, light-scattering, and other techniques described herein. Preferred techniques are visual inspection of a cell-containing medium to determine whether the medium remains pink in the presence of colloids, indicating no aggregate or fibril formation, or changes to blue, indicating colloid aggregation indicative of the presence of aggregate-forming species, or any color indication on the spectrum between pink and blue indicative of a corresponding intermediate state.

Another aspect of the invention provides a method of treatment, comprising withdrawing a sample from a patient and subjecting the sample to a composition. The method further comprises determining whether to treat the patient with the composition.

A “sample” can be any patient sample as described previously. “Subjecting the sample to a composition” comprises adding the composition to a sample in any method known to those of ordinary skill in the art. Typically, the sample exists in solution and is subjected to assay conditions. Thus, the sample from the patient can be treated as in any sample suspected of containing a disease, particularly a disease associated with aggregate formation, and the assay conditions can be any conditions relating to any of the assays described herein, e.g. adding to the sample a plurality of binding species capable of binding to an aggregate. In one embodiment, the step of “determining whether to treat the patient with the composition” comprises determining whether the composition is capable in inhibiting aggregate formation. As described previously, inhibition of aggregate formation can be determined visually, via the naked eye or microscopy, or via spectroscopy.

Of course it is understood that the sample can divided into a number of portions and each portion can be subjected to a different composition. Alternatively, more than one sample or sample type can be withdrawn from the patient and the samples or sample types or portions thereof can be subjected to different compositions.

One aspect of the invention also involves using a cell that can produce an aggregate-forming species for a variety of purposes. In one technique, the cell can be exposed to a candidate drug for inhibition of aggregation-associated physiological processes such as neurodegenerative disease, and the potential of materials produced by the cell for formation of aggregates characteristic of neurodegenerative disease can be monitored. This can be useful for determining the effectiveness of the candidate drug for inhibition of the disease. Other techniques can be carried out as well. Use of a cell itself, rather than simply isolating aggregate-forming species produced by the cell and testing those species, allows one to carry out experiments and monitor drug efficacy in real time. This provides several advantages, including techniques that are simple, inexpensive, and rapid. One technique involves monitoring the production, e.g., secretion, of neurodegenerative disease aggregate or fibril-forming species from the cell as a function of time, for example over periods of time including 5 seconds, 30 seconds, and other small-time intervals including any number of minutes up to 1 day, and over longer periods of time including any number of days up to a week or even longer periods including 2 weeks, 3 weeks, etc. Monitoring production of these species by cells as a function of time allows determination of drug activity profiles as material is continually produced over time, i.e. ability of the drug to continually act in response to newly-produced material. Drug efficacy as a function of time also can be studied. This can allow determination of the effectiveness of the drug in acting in response to different materials produced by the cell at different points in time, for example points in time as described above. Also, stability (thermal) of the drug as a function of time can be determined, as can capacity of the drug to act upon increasing levels of materials secreted by the cell, etc.

Certain methods of the invention need not be limited to cells that have the capability to secrete aggregate-forming species. Alternatively, cells that produce or retain aggregate-forming species intracellularly, can be lysed prior to colloid addition. Although this aspect of the invention is described for a neurodegenerative disease, it applied to non-neurodegenerative disease, and other non-disease processes; the cell may have the ability to produce or retain any aggregate-forming species or precursor of an aggregate-forming species.

In addition, assays can be constructed in different ways to derive information at different points in time relative to production of material in the cell. For example, a cell can be made to produce aggregate-forming species in the presence of a pre-mixed assay including colloids along with binding species fastened or fastenable to the colloids, or colloids and/or binding species can be added at any point in time in the process.

The technique also allows for the determination of the effectiveness of a candidate drug in affecting aggregate formation, where a specific mechanism associated with the formation may not be known. A candidate drug may be determined to be effective in affecting aggregate formation by affecting the production of the species by the cell, or affecting aggregate formation after aggregate forming species have been produced, or affecting of any stage of the process. Stages of the process can include transcription, translation, post-translational modification, and secretion.

The technique also allows for the monitoring, e.g. visualization, of the effectiveness of a candidate drug in affecting aggregate formation over time without intervention. In the assay described, no translation of any assay component, such as pipeting, agitation, or transfer of solution, is required. This can more effectively simulate the natural, biological environment, and thereby more effectively identify drugs that are effective in a natural environment. In this aspect of the invention any of the determination/visualization techniques described herein can be used, e.g. visualization with the unaided human eye of colloid/colloid aggregation, spectrophotometry, light-scattering, and other techniques described herein. Preferred techniques are visual inspection of a cell-containing medium to determine whether the medium remains pink in the presence of colloids, indicating no aggregate or fibril formation, or changes to blue, indicating colloid aggregation indicative of the presence of aggregate-forming species, or any color indication on the spectrum between pink and blue indicative of a corresponding intermediate state.

Another aspect of the invention provides a kit comprising a composition, as described herein. Such a kit may comprise a carrier means being compartmentalized to receive in close confinement one or more container means such as vials, tubes, and the like, each of the container means comprising one of the separate elements to be used in the method. For example, one of the container means may comprise a positive control in the assay. Additionally the kit may include containers for buffer(s) useful in the assay.

Examples of well-known carriers include glass, polystyrene, polypropylene, polyethylene, dextran, nylon, amylase, natural and modified cellulose, polyacrylamide, agarose and magnetite. The nature of the carrier can be either soluble of insoluble for purposes of the invention. Those skilled in the art will know of other suitable carriers for binding peptides, or will be able to ascertain such, using routine experimentation.

In one embodiment, the invention relates to various classes of structures which encompass a composition of the invention for treatment of a patient susceptible to or exhibit symptoms of Alzheimer's Disease. Such classes of structures include:

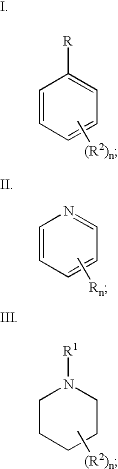

IV. a five membered ring including at least one heteroatom;

V. at least two rings bridged by at least one atom;

VI. at least two rings bonded directly to each other;

VII. a ring containing at least one carbonyl; and

VIII. an alkyl chain.

Referring to classes I-III, R and R² can be the same or different and each is a substituent of an organic group or chlorine. n is an integer greater than or equal to 0 and no more than 6. Within a particular composition, each individual R or R² substituent can be the same or different. R¹ can be a hydrogen or an organic group. R, R¹ and R² of classes I-III and any substituent of the remaining classes are arranged so as to create polarity in the structure. In one embodiment, the polarity is sufficient such that the structure includes a hydrophobic portion and a hydrophilic portion. For compositions including a ring, preferably each substituent is either an electron-donating or electron-accepting group relative to a central ring.

For any of classes I-VII, any ring structure optionally includes at least one other heteroatom including a carbonyl, nitrogen, oxygen, sulfur and silicon. A ring structure including a carbonyl incorporates the carbonyl carbon in the ring. A ring structure including nitrogen, oxygen, silicon or sulfur results in a heterocycle.

Any ring structure optionally includes at least one carbon-carbon double bond within the ring. Class I and II compositions are aromatic inherently include three carbon-carbon double bonds. Class III compositions can include zero, one or two carbon-carbon double bonds within the ring.

Where the drugs contain a stereocenter, in some embodiments, the preferred drugs encompass both enantiomers of the compositions, either individually in pure form or as a racemic mixture. In other embodiments, one enantiomer may be preferred over the other enantiomer.

Compounds can be screened by assays having the general steps of:

1. providing a plurality of particles each having a surface and a plurality of binding species capable of binding a neurodegenerative disease aggregate-forming or fibril-forming species fastened to or adapted to be fastened to the surface;

2. exposing the particles and the binding species to a candidate drug suspected of inhibiting neurodegenerative disease fibril or aggregate formation; and

3. determining an observable feature of the particles indicative of effectiveness of the candidate drug in inhibiting aggregate or fibril formation.

This technique for determining effectiveness of a candidate drug as a function of time helps to indicate whether the drug is effective at an early stage of the disease process, an intermediate stage, a late stage, or at several stages. The observable features of the particles can include visibly-identifiable aggregation, color change (or relative color change, lack of color change, or any identifiable feature involving color), or the like. Most of the features are observable by the unaided human eye. CCD cameras, spectrophotometers, and the like also can be used to determine activity of the particles indicative of effectiveness of the candidate drug. Determination at various points in time can include very early-stage, such as within a period of a few seconds, up to long periods of time such as two days or more. Essentially all periods of time in between can be useful in indicating effectiveness of candidate drugs for various disease stages.

It is a feature of an embodiment of the invention that assays can be conducted to determine effectiveness of drug candidate at various stages without any disruption of samples of the assay. “Disruption”, in this context, means withdrawal of samples, mixing, addition of reagents, shaking, vortexing, or any other activity other than allowing the sample to remain essentially motionless. It is also a feature that assays can be monitored without exposure of the assay to any external sources of energy. “External sources of energy”, in this context, includes heat (above room temperature—about 23° C.), radiation, or agitation as described above, but does not include minimal, periodic exposure to electromagnetic radiation for spectrophotometric determination.

The assays can also involve providing a plurality of particles each having a surface and a plurality of binding species capable of binding a neurodegenerative disease aggregate-forming or fibril-forming species fastened to or adapted to be fastened to the surface. The particles are exposed to a sample containing a neurodegenerative disease aggregate-forming or fibril-forming species in the presence of a candidate drug suspected of inhibiting neurodegenerative disease fibril or aggregate formation. An extent of an observable feature of the particles, such as aggregation, color change, or the like, is determined indicative of effectiveness of the candidate drug in inhibiting aggregate or fibril formation no sooner than five hours after the exposing step in one embodiment. This technique can be used primarily for determining drugs effective in treatment of later-stage processes involved in neurodegenerative disease. In other embodiments, the determining step can be conducted no sooner than ten hours after the exposing step, or fifteen hours, twenty hours, at least one day, at least two days, or longer after the exposing step.

The assays can also involve techniques identical to that described in the paragraph immediately above, except that an observable feature of the particles is determined no later than one minute after the exposing step. In other embodiments the observable feature is determined no later than thirty seconds, twenty seconds, or even ten seconds after the exposing step. This method can be particularly useful in identifying compositions used for treatment of early-staged disease processes.

The time scale of the previously mentioned assays can be shortened or lengthened by altering a variety of conditions such as neurodegenerative disease aggregate or fibril-forming species concentration; presence or absence of pre-formed seed fibrils or aggregates present in the assay; presence, absence, or concentration of auxiliary agents at the surfaces of the particles or free in solution that can accelerate or inhibit aggregation, such as glycol units; and/or exposure of the assay to energy such as agitation, electromagnetic radiation, etc. Thus, the specific periods of time necessary for determination of the effectiveness of various compositions for treatment for various disease stages can vary.

In one embodiment, the composition is a class I composition comprising the structure:

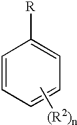

wherein R and R² is selected from the group consisting of a primary amine, a secondary amine, a hydroxyl, an amide, an alkylamide, a carboxyl, a carboxylic acid, a carboxylate, an ester, a sulfonate, a C₁-C₆ alkoxide optionally interrupted or terminated by a chlorine atom or a nitrogen atom and a C₁-C₆ alkyl optionally interrupted or terminated by a chlorine atom or a nitrogen atom. A C₁-C₆ alkoxide or alkyl optionally interrupted or terminated by a chlorine atom or a nitrogen atom can also include an amine, amide or ammonium salt in the middle or terminus of an alkyl or alkoxide chain.

An example of a composition of the present invention in which n=0 is 1-phenyl biguanide.

In one embodiment, the composition is a di-substituted aromatic having the structure:

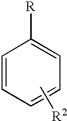

If R and R² have an ortho- or meta-arrangement, R and R² can be the same or different to cause polarity. If R and R² are para to each other, R and R must be different to cause polarity.

Examples of compositions having a meta-arrangement include phenylephrine hydrochloride and arecoline hydrobromide. Examples of drugs having a para-arrangement include S(−)-atenolol, R(−)-atenolol, tetracaine hydrochloride, octopamine hydrochloride and procainamide hydrochloride.

In yet another embodiment, n is greater than 2 such that the composition comprises tri-, tetra-, penta- and even hexa-substituted aromatics. Examples of tri-substituted compositions include (±)-sulpiride, (±)-vanillyl mandelic acid, (−)-α-methyl norepinephrine, normetanephrine hydrochloride, MHPZ piperazine and erbstatin analog.

Many of the aromatic compositions are substituted with alkoxides optionally interrupted or terminated by a nitrogen atom. Certain compositions comprise a tri-alkoxide aromatic (i.e. n=2, R═R²=alkoxide optionally interrupted or terminated by a nitrogen atom), such as gallamine triethiodide.

In another set of embodiments the invention provides a composition defined by a multi-cyclic organic composition of at least two, fused rings where the structure, via incorporation of differences within the rings and/or differences in pendant groups on the rings, exhibits polarity sufficient to define hydrophilic and hydrophobic sections of the molecule. The fused rings can be the same or different and, in a preferred embodiment the rings are different and some of the polarity of the molecule is caused by the differences in the ring structures.

In one subset of this set of embodiments the composition comprises an aromatic structure which is a first ring, and R and at least one of R² is positioned on an adjacent carbon atom. The adjacent R² and R groups comprise a second ring fused to the first ring such that the drug comprises a fused ring structure. The second ring can be a five-, six- or seven-membered ring and can be aromatic or non-aromatic. The second ring can include a heteroatom such as a carbonyl in which the carbonyl carbon is in the ring, nitrogen, sulfur or oxygen. If the second ring is aromatic, it can include any combination of zero, one or two nitrogen atoms and/or one or more heteroatoms. A non-aromatic ring can also include one or two carbon-carbon double bonds.

An example of a fused ring structure having a second ring with a nitrogen atom includes pindolol.

The fused ring can include three, four or more rings fused sequentially, e.g. tri-, tetra- or multi-fused rings. The third or fourth ring can have any of the properties of the second ring, as described previously. Examples of tri-fused ring compositions include (−)-physostigmine and telenzepine dihydrochloride and examples of tetra-fused ring compositions include podophyllotoxin and spironolactone.

In one embodiment, the composition of the invention is a class II composition (i.e. a pyridine derivative) comprising the structure:

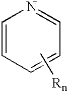

R can be any group as described previously for class I compositions. In a preferred embodiment, R can be a primary amine, a secondary amine, a hydroxyl, a C₁-C₆ alkyl, a C₁-C₆ alkoxide, a carboxyl, a carboxylate, a carboxylic acid and an amide. An example of a class II composition is 4-aminopyridine.

The pyridine derivative can also be fused to a second ring having any of the properties of the second ring of Class I aromatic compounds, as described previously.

In one embodiment, the composition of the invention is a class III composition comprising the structure:

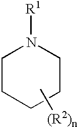

Such non-aromatic compositions can include an additional nitrogen and/or one or more heteroatoms, as described previously. The composition can include one or two carbon-carbon double bonds. An example of a class III composition including a sulfur and a carbonyl group includes chlomezanone. An example of a class III composition having two nitrogen atoms includes uracil, 5-trifluoromethyl-5,6-dihydro, primidone and urapidil-5-methyl, both of which also include carbonyl carbon atoms in the ring.

This composition can also be fused to a second ring having any of the properties of the second ring of Class I aromatic compounds, as described previously. An example of such a fused-ring structure includes debrisoquin sulfate. Examples of fused-ring structures having two nitrogen atoms in each ring include 8-cyclopentyl-1,3-dimethylxanthine and 1-allyl-3,7-dimethyl-8-p-sulphophenyl-xanthine.

In one embodiment, the composition of the invention is a class IV compound comprising a five-membered ring containing at least one heteroatom. Examples of such heteroatoms include oxygen, nitrogen, sulfur and silicon.

In one embodiment, the heteroatom is nitrogen. Examples of such five-membered rings containing at least one nitrogen atom include:

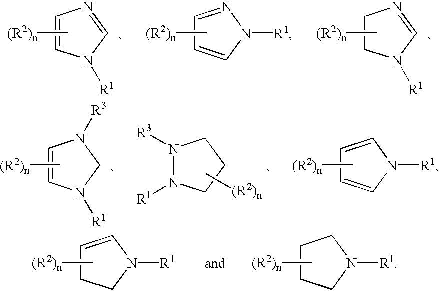

wherein R² is selected from the group consisting of an organic group and chlorine and each R² can be the same or different. n is an integer no more than 4. R¹ and R³ can be the same or different and can be an organic group and hydrogen. In certain embodiments, R¹ and R³ can be a hydrogen, a sugar, a sugar derivative, a C₁-C₆ alkyl optionally interrupted or terminated by a nitrogen, and an aryl. Example compositions include histamine-R(−)-α-methyl-dihydrochloride, histamine-1-methyl-hydrochloride and cimetidine.

The five-membered ring can also include any heteroatom described previously. Examples of five-membered rings including carbonyl carbon atoms include phenylbutazone and oxotremorine methiodide.

Any two R² groups on adjacent carbon atoms are capable of forming a ring such that the drug comprises a fused ring structure. Thus, the five-membered ring can be fused to a ring having any of the properties of the second ring for class I compounds as described previously. In one embodiment, the second ring is a six-membered ring. The six-membered ring can be aromatic, and examples of such fused ring structures include 1,3-dihydro-1-[2-hydroxy-5-(trifluoromethyl)phenyl]-5-(trifluoromethyl)-2H-benzimidazol-2-one. The six-membered ring can have one or two nitrogen atoms, and examples of rings having two nitrogen atoms include N6-cyclopentyl 9-methyladenine, purines and purine derivatives such as DNA bases or DNA base derivatives.

In one embodiment, R¹ or R³ comprises a sugar or sugar derivative and examples include S-(4-nitrobenzyl)-6-thioinosine, S-(4-nitrobenzyl)-6-thioguanosine, N6-methyladenosine and 2-phenylamineadenosine. The sugar or sugar derivative can be bonded to a chain of at least two phosphates and examples include p,p-di(adenosine-5′)-tetraphosphate triammonium.

In one embodiment, the composition is a class V composition having at least two rings bridged by at least one atom such as carbon, nitrogen and sulfur. In certain embodiments, at least two rings are bridged by a bridging unit including a nitrogen atom, —N(R)—, —C(O)N(R)—, —N═N—, a C₁-C₆ alkyl, —C(S)N(R)—, —C(O)R— and —S(O)₂N(R)—, wherein R is selected from the group consisting of an aryl, an aryl radial, a C₁-C₆ alkyl optionally terminated by an amine, a C₁-C₆ alkyl radical and a hydrogen. Examples of such compositions include diclofenac sodium, 4-[[4-formyl-5-hydroxy-6-methyl-3-[(phosphonooxy)methyl]-2-pyridinyl]azo]-1,3-benzenedisulfonic acid tetrasodium salt, phentolamine mesylate, 3,3′,4,4′-tetramethoxy-N-methyl-diphenethylamine hydrochloride, thioperamide maleate and BRL 37344 sodium.

Compositions were identified that inhibit aggregation characteristic of neurodegenerative disease at high B-amyloid peptide concentration, low peptide concentration, and in some cases both. Compositions that inhibited aggregation at high concentration inhibited formation where an assay included B-amyloid peptide and at least some amount of pre-formed aggregate characteristic of neurodegenerative disease. Compositions that inhibited aggregation at low concentration inhibited concentration in the presence of β-amyloid peptide but in the absence of any pre-formed aggregate characteristic of neurodegenerative disease.

The following lists show compositions that inhibited aggregate formation at high peptide concentration, low peptide concentration, including drugs that inhibited aggregate formation at high or low concentration.

Drugs in Bold Inhibited at Both High and Low Peptide Concentration

At High Peptide Concentration:

A-143 S(−)-Atenolol (new to high conc. list)

P-125 pindolol

H-128 histamine, R(−)-α-methyl, dihydrochloride

A-142 R(−)+atenolol

A-134 4-aminopyridine

P-155 (−)-physostigmine

T-114 Tetracaine Hydrochloride

A-025 1-methyl-isoguanosine

C-192 chlomezanone

D-146 debrisoquin sulfate

N-127 s-(4-nitrobenzyl)-6-thioinosine

P-121 phenylbutazone

C-1028-cyclopentyl-1,3-dimethylxanthine

D-151 p,p-di(adenosine-5′)-tetraphosphate triammonuim

N-128 s-(4-nitrobenzyl)-6-thioguanosine

P-141 podophyllotoxin

D-174 diclofenac sodium

M-101 n6-methyladenosine

N-154 n6-cyclopentyl 9-methyladenine

P-171 primidone

A-145 1-allyl-3,7-dimethyl-8-p-sulphophenyl-xanthine

P-178 PPADS (4-[[4-formyl-5-hydroxy-6-methyl-3-[(phosphonooxy)methyl]-2-pyridinyl]azo]-1,3-benzenedisulfonic acid tetrasodium salt)

P-101 2-phenylamineadenosine

V-103 Vanillyl mandelic acid

S-141 Spironolactone

V-107 valproic acid sodium

M-133 (−)-α-methyl norepinephrine

P-133 phenylephrine hydrochloride

U-101 urapidil, 5-methyl

P-131 Phentolamine mesylate

Y-101 YS-035 hydrochloride (3,3′,4,4′-tetramethoxy-N-methyl-diphenethylamine hydrochloride)

H-137 histamine, 1-methyl-hydrochloride

T-123 thioperamide maleate

N-111 normetanephrine hydrochloride

O-101 Octopamine hydrochloride

G-104 gallamine triethiodide

O-100 oxotremorine methiodide

P-137 Procainamide hydrochloride

T-122 telenzepine dihydrochloride

C-006 arecoline hydrobromide

S-116 sulpiride

U-114 uracil, 5-trifluoromethyl-5,6-dihydro

P-120 1-phenylbiguanide

A-001 7-(β-chloroethyl)-theophylline

L-102 lidocaine hydrochloride

E-114 erythro-9-(2-hydroxy-3-nonyl)adenine hydrochloride

M-116 metolazone

M-128 methylene adenosine 5′-triphosphate dilithium

A-022 1,3-dipropyl-8-p-sulfophenyl xanthine

M-152 2-methyl-thioadenosine diphosphate trisodium

A-202 n6-2-(4-amino-phenyl) ethyladenosine

G-018 2-amino-7-phosphonoheptanoic acid

H-1743-hydroxy-phenylglycine

M-172 methyl-4-carboxy-phenyl glycine

F-123 Fusaric acid

S-147 sulphaphenazole

M-134 methoxamine hydrochloride

U-104 UK14304 (5-bromo-N-(4,5-dihydro-1-H-imidazol-2-yl)-6-quinaxalinamine)

A-013 8-(p-sulfophenyl)-theophylline

P-016 isoxanthopterin

N-114 Nifedipine

T-125 Trihexylphenidyl hydrochloride

T-111 TMB-8-hydrochloride (8-(N,N-diethylamino)octyl 3,4,5-trimethoxybenzoate hydrochloride)

N-110 Neostigmine bromide

1-104 Isoproterenol hydrochloride

N-153 Nylidrin hydrochloride

U-100 Urapidil hydrochloride

T-141 Tripelennamine hydrochloride

At Low Peptide Concentration

S-116 sulpiride

U-1 14 uracil, 5-trifluoromethyl-5,6-dihydro

A-143 S(−)-Atenolol

P-125 pindolol

B-169 BRL37344 sodium

B-017 MHPZ piperazine (2:1 ratio of 1-(4-hydroxy-3-methoxyphenyl)-1,2-ethanediol and diethyldiazene)

A-142 R(−)+atenolol

C-118 cimetidine

M-133 (−)-a-methyl norepinephrine

O-100 oxotremorine methiodide

P-155 (−)-physostigmine

A-134 4-aminopyridine

N-170 NS-1619 (1,3-dihydro-1-[2-hydroxy-5-(trifluoromethyl)phenyl]-5-(trifluoromethyl)-2H-benzimidazol-2-one)

E-120 erbstatin analog

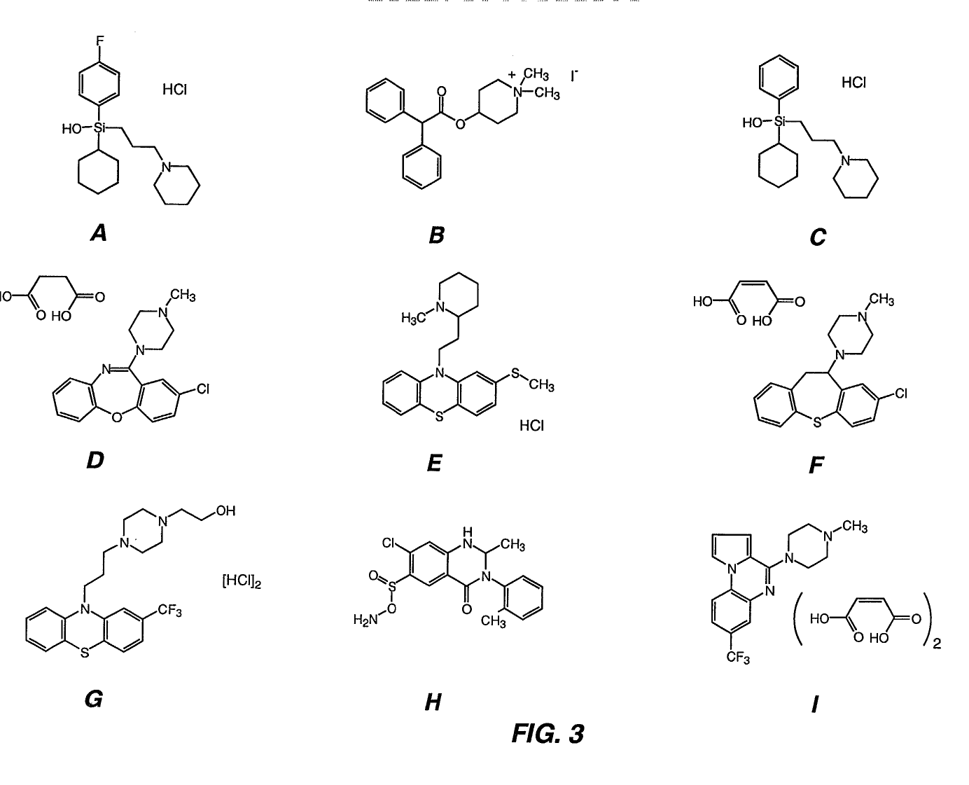

Structures of the above-listed compositions are shown in FIGS. 3-10.

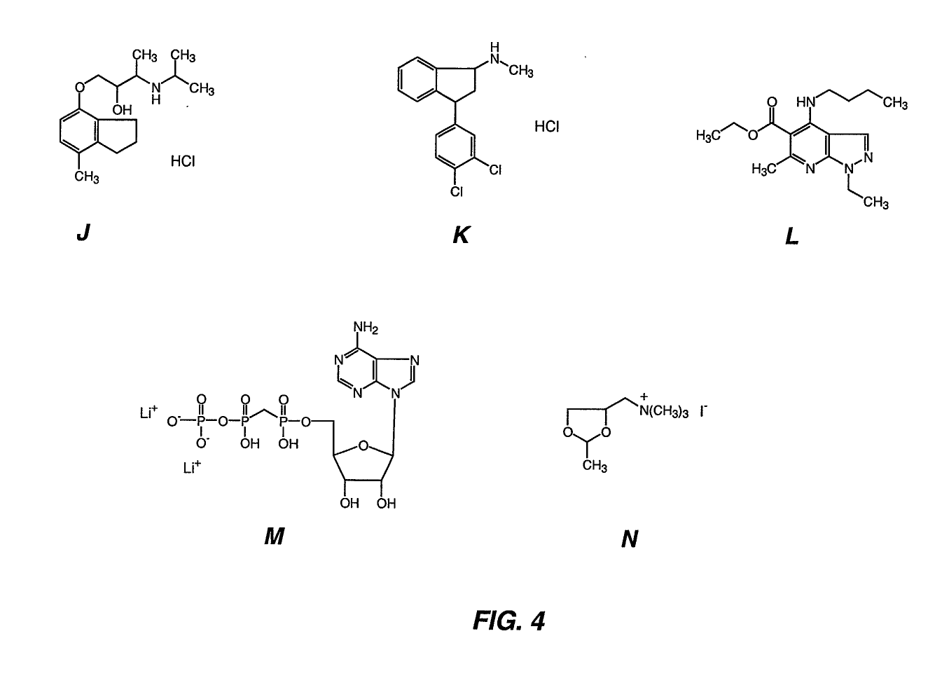

The names of structures A-N shown in FIGS. 3 and 4 are listed below:

A. p-fluorohexahydro-sila-difenidol hydrochloride

B. 4-DAMP methiodide

C. hexahydro-sila-difenidol hydrochloride

D. Loxapine succinate

E. Thioridazine hydrochloride

F. (+/−) octoclothepin maleate

G. luphenazine dihydrochloride

H. Metazone

I. CGS-12066A dimaleate

J. ICI 11,551 hydrochloride

K. indatraline hydrochloride

L. Tracazolate

M. alpha, beta-methylene adenosine 5′ triphosphate dilithium

N. (+)-cis-dioxolane

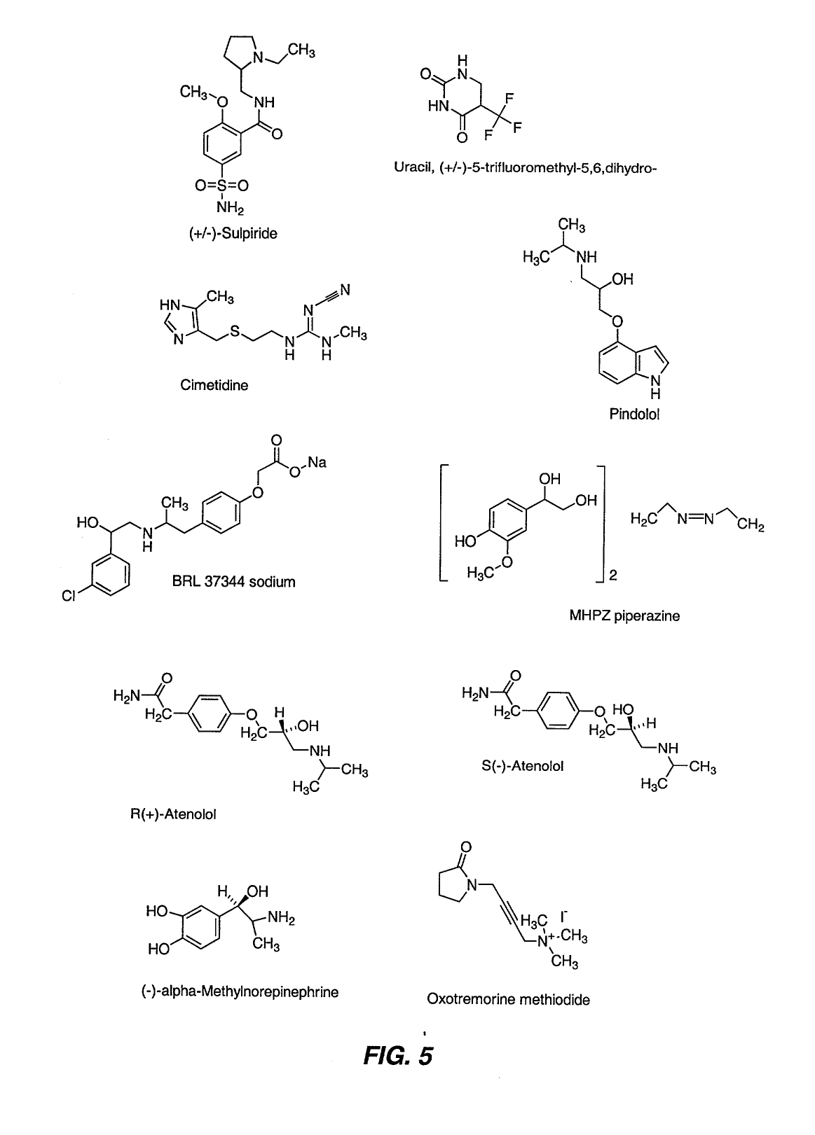

FIG. 5 shows structures of sulpiride, uracil, 5-trifluoromethyl-5,6-dihydro, cimetidine, pindolol, BRL 37344 sodium, MHPZ piperazine (2:1 ratio of 1-(4-hydroxy-3-methoxyphenyl)-1,2-ethanediol and diethyldiazene), R(−)+atenolol, S(−)-atenolol, (−)-α-methyl norepinephrine and oxotremorine methiodide.

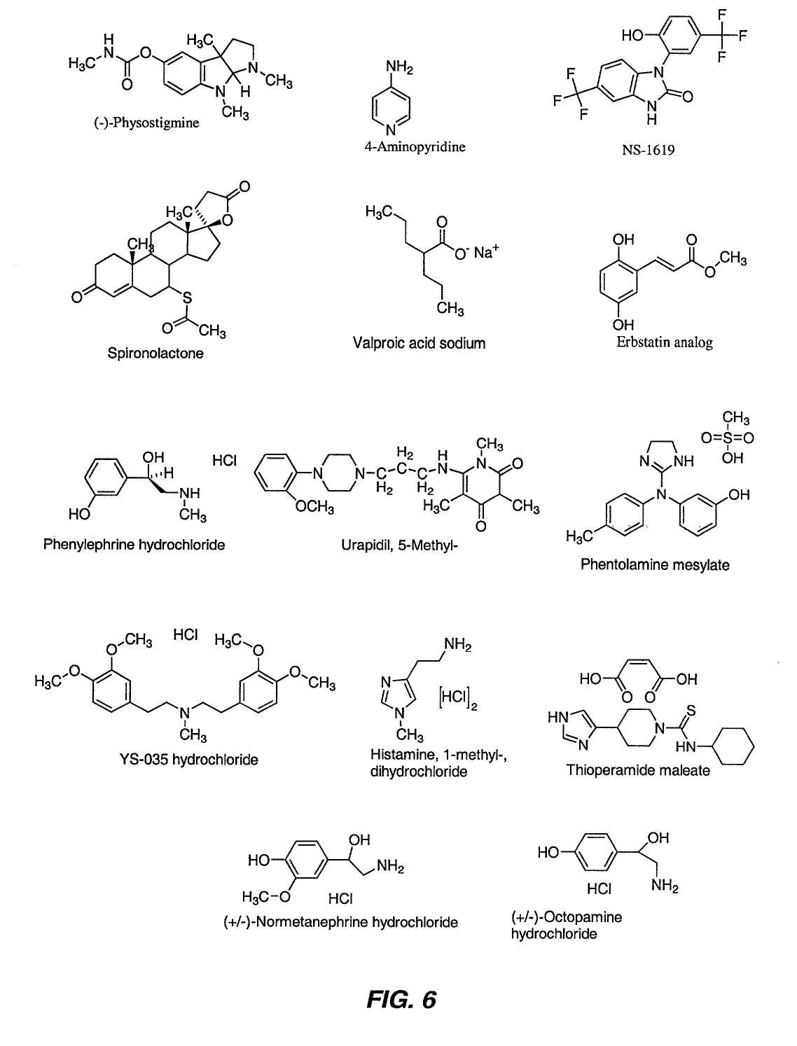

FIG. 6 shows structures of 4-aminopyridine, NS-1619 (1,3-dihydro-1-[2-hydroxy-5-(trifluoromethyl)phenyl]-5-(trifluoromethyl)-2H-benzimidazol-2-one), (−)-physostigmine, erbstatin analog, spironolactone, valproic acid sodium, phenylephrine hydrochloride, urapidil, 5-methyl, phentolamine mesylate, YS-035 hydrochloride (3,3′,4,4′-tetramethoxy-N-methyl-diphenethylamine hydrochloride), histamine, 1-methyl-dihydrochloride, thioperamide maleate, normetanephrine hydrochloride and octopamine hydrochloride.

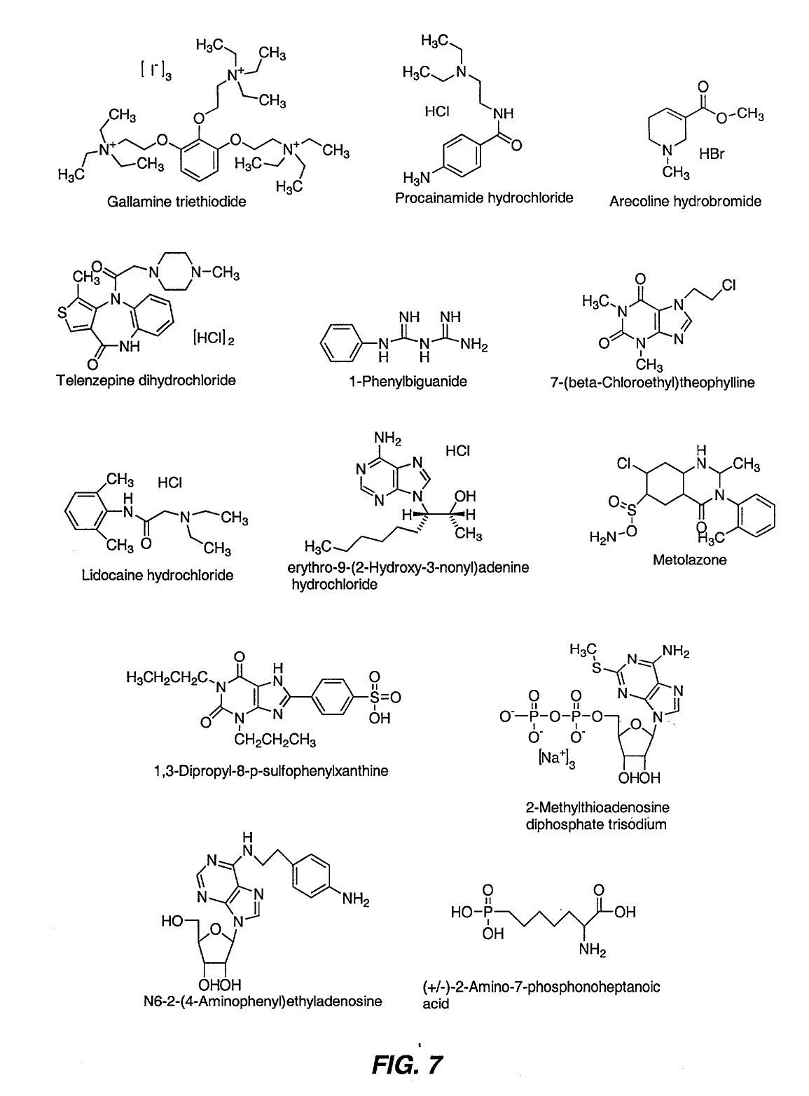

FIG. 7 shows structures of gallamine triethiodide, procainamide hydrochloride, arecoline hydrobromide, telenzepine dihydrochloride, 1-phenylbiguanide, 7-(β-chloroethyl)-theophylline, lidocaine hydrochloride, erythro-9-(2-hydroxy-3-nonyl)adenine hydrochloride, metolazone, 1,3-dipropyl-8-p-sulfophenyl xanthine, 2-methyl-thioadenosine diphosphate trisodium, N6-2-(4-amino-phenyl) ethyladenosine and 2-amino-7-phosphonoheptanoic acid.

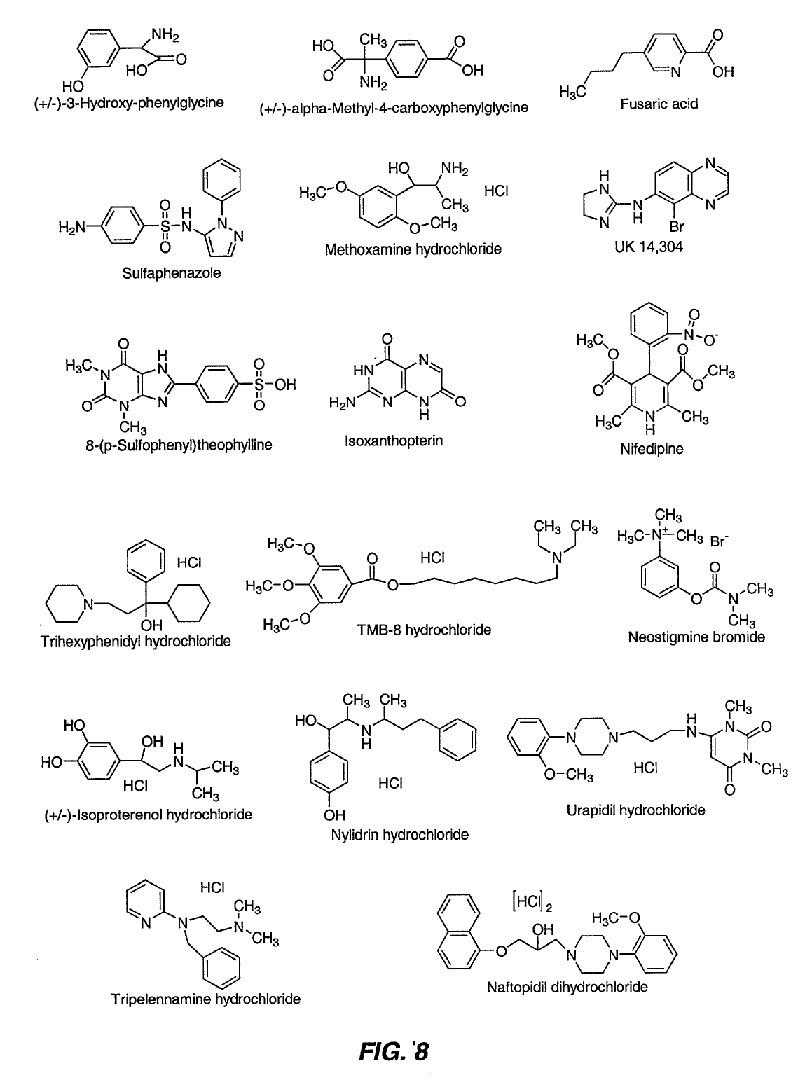

FIG. 8 shows structures of 3-hydroxy-phenylglycine, methyl-4-carboxy-phenyl glycine, fusaric acid, sulphaphenazole, methoxamine hydrochloride, UK14304 (5-bromo-N-(4,5-dihydro-1-H-imidazol-2-yl)-6-quinaxalinamine), 8-(p-sulfophenyl)-theophylline, isoxanthopterin, nifedipine, trihexylphenidyl hydrochloride, TMB-8-hydrochloride (8-(N,N-diethylamino)octyl 3,4,5-trimethoxybenzoate hydrochloride), neostigmine bromide, isoproterenol hydrochloride, nylidrin hydrochloride, urapidil hydrochloride, tripelennamine hydrochloride and naftopidil dihydrochloride.

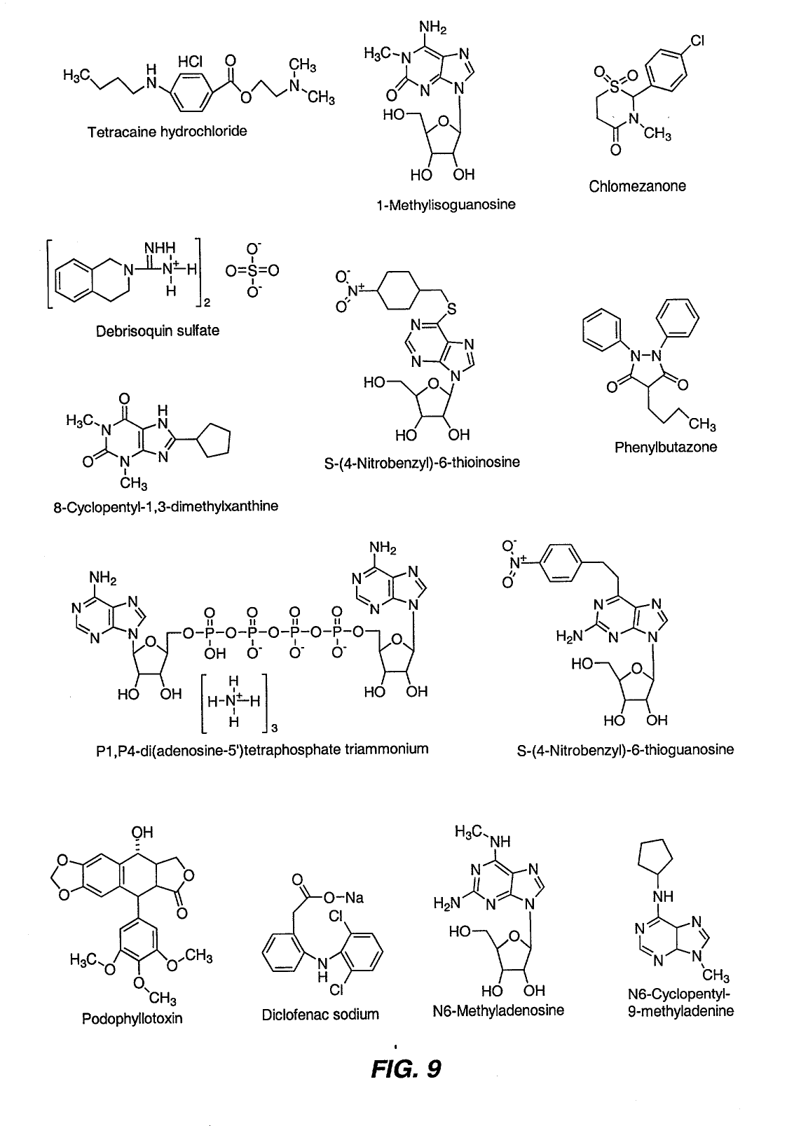

FIG. 9 shows structures of tetracaine hydrochloride, 1-methyl-isoguanosine, chlomezanone, debrisoquin sulfate, 8-cyclopentyl-1,3-dimethylxanthine, S-(4-nitrobenzyl)-6-thioinosine, phenylbutazone, p,p-di(adenosine-5′)-tetraphosphate triammonuim, S-(4-nitrobenzyl)-6-thioguanosine, podophyllotoxin, diclofenac sodium, N6-methyladenosine and N6-cyclopentyl 9-methyladenine.

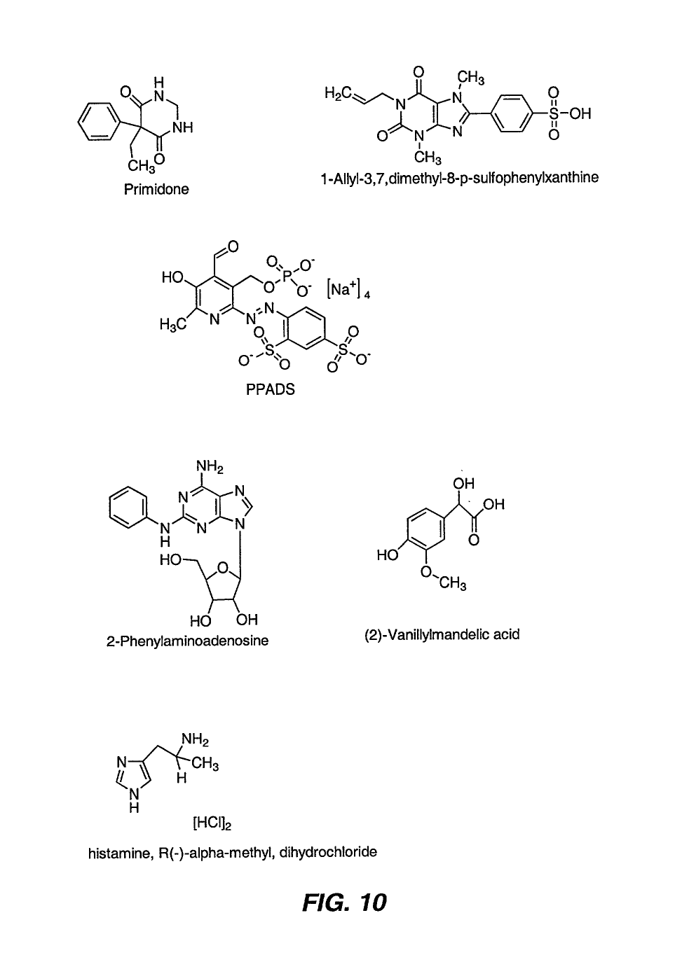

FIG. 10 shows structures of primidone, 1-allyl-3,7-dimethyl-8-p-sulphophenyl-xanthine, PPADS (4-[[4-formyl-5-hydroxy-6-methyl-3-[(phosphonooxy)methyl]-2-pyridinyl]azo]-1,3-benzenedisulfonic acid tetrasodium salt), 2-phenylamineadenosine, vanillyl mandelic acid and histamine, R(−)-α-methyl, dihydrochloride.

It is understood that any of these structures can fall within one or more classes of compositions described above.

One subset of compositions of interest are DNA bases, RNA bases, and analogs or derivatives thereof. In one embodiment, the composition comprises an anti-metabolite, specifically those antimetabolites used as chemotherapy agents.

In one embodiment, a preferred composition comprises at least one aromatic ring comprising any number of substituents directly bonded to the ring in which the ring has a net polarization, or polarity. The net polarization of any aromatic ring can be determined by taking into account the number of substituents, the electronegativity (i.e. electron donating/withdrawing capability) of each substituent and the substitution pattern of the aromatic ring. For example, a benzene ring having two of the same substituents in the para positions will have a net polarization of zero because there is no net dipole. The number of substituents and/or substitution pattern (e.g. symmetric or nonsymmetric) can have an effect on the net polarization. For example, a benzene ring with only one substituent, three substituents or two substituents in ortho or meta positions, will generally have a net polarization regardless of the electronegativity of the substituent. The electronegativity of the substituent can also have an effect on the net polarization. For example, a para-substituted benzene ring having one electron withdrawing group and one electron donating group will have a net polarization. Electronegativity values are available in a number of reference sources, as known by those of ordinary skill in the art.

The aromatic ring is not limited to a benzene ring, and can comprise any closed ring, carbon-based or heterocyclic, having 4 n+2 pi electrons, where n is an integer. The ring can comprise any number of atoms so long as a closed ring is formed.

In one embodiment, electronegativity can be approximated by determining whether the substituent is electron withdrawing or electron donating with respect to a hydrogen atom. Assuming a hydrogen atom has an electronegativity of zero, a substituent that is electron withdrawing with respect to hydrogen has a negative “I” value, i.e. I=(−), whereas a substituent that is electron donating with respect to hydrogen has a positive “I” value, i.e. I=(+). Examples of substituents where I=(−) have an electron-withdrawing atom that is directly bonded to the aromatic ring, such as oxygen or nitrogen, or a carbon atom further bonded to an aromatic group. Examples of substituents where I=(+) include a carbon atom that is directly bonded a non-aromatic ring, such as an alkyl group or a carboxyl group.

Examples of a highly polarized aromatic ring include a benzene ring having two or three oxygen atoms directly bonded to three successive benzene carbon atoms in which the other benzene carbon atoms remain unsubstituted (e.g. gallamine triethiodide) or are substituted with one or more I=(+) groups (e.g. TMB-8 hydrochloride). Aromatic structures which do not provide a positive result in any one of the assays of the invention (and are thus not a composition of the invention) include trimethoprim. Trimethoprin has a benzene ring with the 3- ,4- and 5-positions of the benzene ring are directly bonded to methoxy groups and the 1-position is an alkylaromatic group. In general, the compositions described herein as being a composition of the invention comprise an aromatic group that is highly polarized.

Another aspect of the present invention involves a method comprising providing any of the structures as disclosed herein or as determined from any of the assays described herein and performing a combinatorial synthesis on any one of those structures, preferably to obtain a derivative of the composition. For example, the effectiveness of a composition can be enhanced if it had greater polarity. Thus, the composition is reacted with a variety of electron donating or withdrawing groups in a combinatorial fashion to obtain a composition (i.e. derivative) of greater polarity. An assay is performed with the derivative to determine its effectiveness in inhibiting neurodegenerative disease. The combinatorial synthesis can involve subjecting a plurality of the compositions described herein to combinatorial synthesis.

One aspect of the invention provides a method for treating a subject having a disease associated with fibril formation with any of the compositions described herein and administering to the subject the composition in an amount effective to inhibit, prevent or reduce aggregation. The subject is otherwise free of symptoms calling for treatment with the composition, and one of ordinary skill in the art can readily determine prior uses of the composition from any number of resources, including Harrison's Principles of Internal Medicine, Isselbacher, McGraw Hill, New York (1994).

The compositions disclosed herein, particularly those listed previously, also encompass homologs, analogs, derivatives, variants, functionally equivalent fragments and enantiomers thereof. Such homologs, analogs, derivatives, variants, functionally equivalent fragments and enantiomers can be identified by any of the assays described above which screen for inhibiting fibril formation. “Functionally equivalent” refers to a composition that is capable of treatment of patients having such diseases, or of patients susceptible to these diseases. Alternatively, “functionally equivalent” refers to a composition capable of preventing or treating diseases associated with fibril formation. It will be understood that the skilled artisan will be able to manipulate the conditions in a manner to prepare the homologs, analogs, derivatives, variants, functionally equivalent fragments and enantiomers of the compositions disclosed herein.

Subjects for whom the treatment methods of the invention with sulpiride are not intended are those who are diagnosed with conditions which already call for treatment with sulpiride, such as psychosis. Sulpiride is a dopamine D2 antagonist and is used as an antipsychotic and/or antidepressent agent. Sulpiride has been used to treat schizophrenia and other neurotic disorders with symptoms of anxiety, mixed anxiety-depression, tension, obsessions and hypochondriasis. Sulpiride has been administered orally in a dosage of 200 to 1000 mg daily for the treatment of Tourette syndrome. Other previously known uses for sulpiride include the enhancement of the efficacy of antacids (aluminum-magnesium hydroxide) on duodenal ulcer healing. It is known in the art to administer a combination of sulpiride with at least one other agent to treat Alzheimer's patients, but where sulpiride is used for its antipsychotic properties or as an antidepressant (the other agent(s) is directed to treatment of Alzheimer's itself). Specifically, the prior art includes a teaching that sulpiride can be used to treat the psychotic aspects of later stage Alzheimer's. Accordingly, one aspect of the invention involves treatment of Alzheimer's with sulpiride, not in combination with another agent where the other agent has been previously taught for use in treatment of Alzheimer's itself. Another embodiment involves treatment of Alzheimer's with sulpiride alone, not in combination with any other active agent. Anther embodiment involves treatment of Alzheimer's with sulpiride where the use of sulpiride in the treatment is specifically instructed (through, e.g. written instructions that can accompany the composition) for the treatment of Alzheimer's. In a preferred embodiment of this aspect, the invention involves treatment of Alzheimer's with sulpiride where the use of sulpiride in the treatment is specifically instructed to reduce, prevent, minimize, or reverse, neurodegenerative aggregate or fibril formation. Subjects for whom treatmetn methods in this aspect of the invention with S(−)−Atenolol and/or R(−)+atenolol are not intended are those who are diagnosed with conditions which already call for treatment with S(−)-Atenolol or R(−)+atenolol. Both S(−)−Atenolol or R(−)+atenolol have been used previously in the treatment of hypertension, the management of angina pectoris due to coronary atherosclerosis and the management of hemodynamically stable patients with definite or suspected acute myocardial infaction to reduce cardiovascular mortality. In one embodiment, the invention involves treatment with non-racemic S(−)−Atenolol. In another embodiment, the invention involves treatment with non-racemic R(−)+atenolol. In another embodiment, the invention involves treatment with atenolol as a racemic mixture.

Subjects for whom methods of treatment with pindolol in this aspect of the invention are not intended are those who are diagnosed with conditions which already call for treatment with pindolol. Pindolol is a beta-adrenoceptor blocker/5-hydroxytryptamine(1A/1B) antagonist. Prior uses for pindolol include treatment of depression by enhancement of the clinical antidepressant response to selective serotonin re-uptake inhibitors, and as a beta-adrenoceptor blocker to decrease blood pressure, treat hypertension, induce vasodilation, and treat heart failure. In one embodiment, the treatment methods of the invention do not encompass the use of pindalol as a 5-hydroxytryptamine(1A/1B) antagonist in combination with a tachykinin receptor antagonist useful in the treatment or prevention of disorders of the central nervous system, as described in U.S. Pat. No. 6,096,742.

Subjects for whom methods of treatment with cimetidine in this aspect of the invention are not intended are those who are diagnosed with conditions which already call for treatment with cimetidine, and such subjects include those requiring short-term treatment of active duodenal ulcer, short-term treatment of active benign gastric ulcer, treatment of erosive gastroesophageal reflux disease, treatment for prevention of upper gastrointestinal bleeding (in critically ill patients), or treatment of pathological hypersecretory conditions. In one embodiment, the invention does not encompass the use of cimetidine in the treatment of a disease, such as Alzheimer's or Parkinson's disease, where cimetidine is used in combination with another drug to take advantage of the function of cimetidine as a histamine receptor antagonist, as described in U.S. Pat. No. 6,172,085.

Subjects for whom methods of treatment with (−)-α-methyl norepinephrine in this aspect of the invention are not intended are those who are diagnosed with conditions which already call for treatment with (−)-α-methyl norepinephrine. α-methyl norepinephrine, generated via the transport of α-methyldopa into the central nervous system and converted into α-methyl norepinephrine by enzymes inside the central nervous system, can act as an active α2 agonist form of a drug to protect against damage caused by n-methyl-D-aspartate receptor hypofunction as occurs in schizophrenia, as described in U.S. Pat. No. 5,605,911.

Subjects for whom methods of treatment with BRL 37344 sodium in this aspect of the invention are not intended are those who are diagnosed with conditions which already call for treatment with BRL 37344 sodium.

Subjects for whom methods of treatment with uracil, 5-trifluoromethyl-5,6-dihydro in this aspect of the invention are not intended are those who are diagnosed with conditions which already call for treatment with uracil, 5-trifluoromethyl-5,6-dihydro.

Subjects for whom methods of treatment with MHPZ piperazine in this aspect of the invention are not intended are those who are diagnosed with conditions which already call for treatment with MHPZ piperazine.

Subjects for whom methods of treatment with oxotremorine methiodide in this aspect of the invention are not intended are those who are diagnosed with conditions which already call for treatment with oxotremorine methiodide.

Subjects for whom methods of treatment with NS-1619 in this aspect of the invention are not intended are those who are diagnosed with conditions which already call for treatment with NS-1619.

Subjects for whom methods of treatment with (−) physostigmine in this aspect of the invention are not intended are those who are diagnosed with conditions which already call for treatment with (−) physostigmine. Previous uses include the treatment of senile dementia of the Alzheimer type (DAT) with a drug in combination with physostigmine in its function as an acetylcholinesterase inhibitor, as described in U.S. Pat. No. 6,124,318. Physostigmine has previously been characterized as having no therapeutic benefit and its use is limited by a narrow range of effective doses (U.S. Pat. Re. No. 34,653).

Subjects for whom methods of treatment with 4-aminopyridine in this aspect of the invention are not intended are those who are diagnosed with conditions which already call for treatment with 4-aminopyridine. 4-aminopyridine is an ion channel modulator. 4-aminopyridine has been suggested for inhibiting the increase of APP synthesis (U.S. Pat. No. 6,184,248). 4-aminopyridine and its derivatives is also known as a toxin for blocking transient outward currents channels present in cell membranes of neurones (U.S. Pat. No. 5,821,251).

Subjects for whom methods of treatment with erbstatin analog in this aspect of the invention are not intended are those who are diagnosed with conditions which already call for treatment with erbstatin analog. Erbstatin analog is an inhibitor of EGF receptor-associated tyrosine kinase and other receptor kinases. It has been suggested for use in treating cancers of epithelial cell growth.

Subjects for whom methods of treatment with histamine, R(−)-α-methyl, dihydrochloride in this aspect of the invention are not intended are those who are diagnosed with conditions which already call for treatment with histamine, R(−)-α-methyl, dihydrochloride.

Subjects for whom methods of treatment with tetracaine hydrochloride in this aspect of the invention are not intended are those who are diagnosed with conditions which already call for treatment with tetracaine hydrochloride.

In one aspect, subjects for whom methods of treatment with spironolactone in this aspect of the invention are not intended are those who are diagnosed with conditions which already call for treatment with spironolactone. Previous uses for spironolactone include short-term preoperative treatment of patients with primary hyperaldosteronism; Long-term maintenance therapy for patients with bilateral micro- and macronodular adrenal hyperplasia (idiopathic hyperplasia); the management of edema and sodium retention for a patient with congestive heart failure, hepatic cirrhosis or nephrotic syndrome, when the patient is only partially responsive to, or is intolerant of, other therapeutic measures; the treatment of essential hypertension, usually in combination with other drugs; and for the treatment of patients with diuretic-induced hypokalemia when other measure are considered inappropriate or inadequate. It is known in the art to administer a combination of spironolactone with at least one other agent (e.g. an angiotensin-II antagonist) to treat Alzheimer's patients. However, in such cases spironolactone is used for its antihypertensive properties or as modulator of dopamine activity, and the inventors are not aware of any teaching of spironolactone for the treatment of Alzheimer's directly, e.g. by using spironolactone alone. Accordingly, one aspect of the invention involves treatment of Alzheimer's with spironolactone, not in combination with another agent where the other agent has been previously taught for use in treatment of Alzheimer's itself. Another embodiment involves treatment of Alzheimer's with spironolactone alone, not in combination with any other active agent. Anther embodiment involves treatment of Alzheimer's with spironolactone where the use of spironolactone in the treatment is specifically instructed (through, e.g. written instructions that can accompany the composition) for the treatment of Alzheimer's. In a preferred embodiment of this aspect, the invention involves treatment of Alzheimer's with spironolactone where the use of spironolactone in the treatment is specifically instructed to reduce, prevent, minimize, or reverse, neurodegenerative aggregate or fibril formation.

The methods in this aspect of the invention do not encompass the administration of 5-fluorouracil to a subject in need of treatment of topical treatment of actinic or solar keratoses or pancreatic cancer.

A subject, as used herein, refers to any mammal (preferably, a human), and preferably a mammal that may be susceptible to diseases or conditions associated with fibril formation. Examples include a human, non-human primate, cow, horse, pig, sheep, goat, dog, cat, or rodent. In some embodiments human subjects are preferred.

The subject can be apparently healthy. An apparently healthy subject is one who at the time of treatment does not exhibit disease symptoms, or at least symptoms associated with fibril formation-type diseases. In other words, such individuals, if examined by a medical professional, would be characterized as healthy and free of symptoms of the disease. The apparently healthy subjects however may still demonstrate particular risk factors which may place them at an elevated risk of diseases or conditions associated with fibril formation. For example, such subjects may be apparently healthy and still have a family history of these diseases. In one embodiment, the treatment is directed to a subject susceptible to or at risk of acquiring diseases or conditions associated with fibril formation.

In some aspects, the invention intends to treat subjects who are at risk of a disease event arising from fibril formation. These subjects may or may not have had a previous this disease event. This invention embraces the treatment of subjects prior to the event, at a time of the disease event, following the disease event, or who have been diagnosed as having the disease. Thus, as used herein, the “treatment” of a subject is intended to embrace both prophylactic and therapeutic treatment, and can be used both to limit or to eliminate altogether the symptoms or the occurrence of the disease event. Some aspects of the present invention also intend to encompass the treatment of a subject that has an abnormally elevated risk of a the event. The subject may already have the disease.

Factors which may predispose subjects to abnormally elevated risk of a diseases associated with fibril formation are genetic risk factors and/or lifestyle habits. Inherited conditions can generally be regarded as pre-disease states. The pre-disease subject can sometimes be identified if they present with a personal history of early (i.e., adolescent or as a young adult) and/or repeated disease events in the absence of an overt pre-disposing condition, and/or a family history of such disease-related conditions. Subjects who have experienced (symptoms), may be regarded as having a personal history of such diseases, and are thus also at risk of a the disease event.

In yet another embodiment, the subject is one who will undergo an elective surgical procedure. The agent may be administered to such a subject prior to the elective surgical procedure. The method of the invention can also be directed towards a subject who has undergone a surgical procedure. As used herein, a surgical procedure is meant to encompass those procedures that have been classically regarded as surgical procedures as well as interventional procedures.

The method comprises administering to the subject a composition of the invention in an amount effective to provide a medically desirable result.

When administered, the compositions of the invention are applied in pharmaceutically acceptable amounts and in pharmaceutically acceptable compositions. Such preparations may routinely contain salts, buffering agents, preservatives, compatible carriers, and optionally other therapeutic ingredients. Such pharmacologically and pharmaceutically acceptable salts include, but are not limited to, those prepared from the following acids: hydrochloric, hydrobromic, sulphuric, nitric, phosphoric, maleic, acetic, salicylic, p-toluene sulfonic, tartaric, citric, methane sulfonic, formic, malonic, succinic, naphthalene-2-sulfonic, and benzene sulfonic. Also, pharmaceutically acceptable salts can be prepared as alkaline metal or alkaline earth salts, such as sodium, potassium or calcium salts of the carboxylic acid group.

In administering the compounds of the invention to subjects, dosing amounts, dosing schedules, routes of administration and the like may be selected so as to affect the other known activities of these compounds. For example, amounts, dosing schedules and routes of administration can be selected as described herein, whereby therapeutically effective levels for inhibiting fibril formation are provided.

The effective amount will vary with the particular condition being treated, the age and physical condition of the subject being treated, the severity of the condition, the duration of the treatment, the nature of the concurrent therapy (if any), the specific route of administration and like factors within the knowledge and expertise of the health practitioner. For example, in connection with diseases associated with fibril formation, an effective amount is that amount which prevents further aggregation, or any such aggregation at all. A medically desirable result can be the inhibition of fibril formation or even a reduction in the size of the aggregate. It is preferred generally that a maximum dose be used, that is, the highest safe dose according to sound medical judgment.

When used therapeutically, the agents of the invention are administered in therapeutically effective amounts. In general, a therapeutically effective amount means that amount necessary to delay the onset of, inhibit the progression of, or halt altogether the particular condition being treated. Generally, a therapeutically effective amount will vary with the subject's age, condition, and sex, as well as the nature and extent of the disease in the subject, all of which can be determined by one of ordinary skill in the art. The dosage may be adjusted by the individual physician or veterinarian, particularly in the event of any complication. A therapeutically effective amount typically varies from 0.01 mg/kg to about 1000 mg/kg. It is expected that does ranging from 1-500 mg/kg, and preferably doses ranging from 1-50 mg/kg will be suitable. In other embodiments, the agents will be administered in doses ranging from 1 μg/kg/day to 10 mg/kg/day, with even more preferred doses ranging from 1-200 μg/kg/day, 1-100 μg/kg/day, 1-50 μg/kg/day or from 1-25 μg/kg/day. In other embodiments, dosages may range from about 0.1 mg/kg to about 200 mg/kg, and most preferably from about 0.2 mg/kg to about 20 mg/kg. These dosages can be applied in one or more dose administrations daily, for one or more days.

Dosages are estimated based on the results experimental models optionally in combination with results of assays of some embodiments of the invention. Generally, daily oral prophylactic doses of active compounds will be from about 0.01 milligrams/kg per day to 2000 milligrams/kg per day. It is expected that oral doses in the range of 10 to 500 milligrams/kg, in one or several administrations per day, will yield the desired results. In the event that the response in a subject is insufficient at such doses, even higher doses (or effective higher doses by a different, more localized delivery route) may be employed to the extent that patient tolerance permits. Multiple doses per day are contemplated to achieve appropriate systemic levels of compounds.

The agent of the invention should be administered for a length of time sufficient to provide either or both therapeutic and prophylactic benefit to the subject. Generally, the agent is administered for at least one day. In some instances, the agent may be administered for the remainder of the subject's life. The rate at which the agent is administered may vary depending upon the needs of the subject and the mode of administration. For example, it may be necessary in some instances to administer higher and more frequent doses of the agent to a subject for example during or immediately following a disease event, provided still that such doses achieve the medically desirable result. On the other hand, it may be desirable to administer lower doses in order to maintain the medically desirable result (e.g. prevent, minimize or cease aggregation) once it is achieved. In still other embodiments, the same dosage may be administered throughout the treatment period which as described herein may extend throughout the lifetime of the subject. The frequency of administration may vary depending upon the characteristics of the subject. The agent may be administered daily, every 2 days, every 3 days, every 4 days, every 5 days, every week, every 10 days, every 2 weeks, every month, or more, or any time therebetween as if such time was explicitly recited herein.

In one embodiment, daily doses of active compounds will be from about 0.01 milligrams/kg per day to 1000 milligrams/kg per day. It is expected that oral doses in the range of 50 to 500 milligrams/kg, in one or several administrations per day, will yield the desired results. Dosage may be adjusted appropriately to achieve desired drug levels, local or systemic, depending upon the mode of administration. In the event that the response in a subject is insufficient at such doses, even higher doses (or effective higher doses by a different, more localized delivery route) may be employed to the extent that patient tolerance permits. Multiple doses per day are contemplated to achieve appropriate systemic levels of compounds.

Preferably, such drugs are used in a dose, formulation and administration schedule which favor the activity of the agent and do not impact significantly, if at all, on normal cellular functions.

The therapeutically effective amount of the agents is that amount effective to inhibit, cease or reduce aggregation in a blood sample, tissue sample or other sample as described by the assays described herein. The samples used herein are any body tissue or body fluid sample obtained from a subject, as described previously. Preferred are body fluids, for example lymph, saliva, blood, urine, and the like. Blood is most preferred. Samples of tissue and/or cells for use in the various methods described herein can be obtained through standard methods including, but not limited to: tissue biopsy, including punch biopsy and cell scraping, needle biopsy, and collection of blood or other bodily fluids by aspiration or other methods.

When administered to subjects for therapeutic purposes, the formulations of the invention are applied in pharmaceutically acceptable amounts and in pharmaceutically acceptable compositions. According to certain methods of the invention, the composition may be administered in a pharmaceutically acceptable carrier. Such a pharmaceutical composition may include the agents of the invention in combination with any standard physiologically and/or pharmaceutically acceptable carriers which are known in the art. The compositions should be sterile and contain a therapeutically effective amount of the agent in a unit of weight or volume suitable for administration to a patient. The term “pharmaceutically-acceptable carrier” as used herein means one or more compatible solid or liquid filler, diluents or encapsulating substances which are suitable for administration into a human or other animal. As used herein, a pharmaceutically-acceptable carrier means a non-toxic material that does not interfere with the effectiveness of the biological activity of the active ingredients. The term “carrier” denotes an organic or inorganic ingredient, natural or synthetic, with which the active ingredient is combined to facilitate the application. The components of the pharmaceutical compositions also are capable of being co-mingled with the molecules of the present invention, and with each other, in a manner such that there is no interaction which would substantially impair the desired pharmaceutical efficacy. Pharmaceutically acceptable further means a non-toxic material that is compatible with a biological system such as a cell, cell culture, tissue, or organism. The characteristics of the carrier will depend on the route of administration. Physiologically and pharmaceutically acceptable carriers include diluents, fillers, salts, buffers, stabilizers, solubilizers, and other materials which are well known in the art.

Pharmaceutically acceptable carriers include diluents, fillers, salts, buffers, stabilizers, solubilizers and other materials which are well-known in the art. The compositions of the invention may be formulated into preparations in solid, semi-solid, liquid or gaseous forms such as tablets, capsules, powders, granules, ointments, solutions, depositories, inhalants and injections, and usual ways for oral, parenteral or surgical administration. Some embodiments of the invention also embrace locally administering the compositions of the invention, including as implants.

Such preparations may routinely contain salts, buffering agents, preservatives, compatible carriers, and optionally other therapeutic ingredients. When used in medicine the salts should be pharmaceutically acceptable, but non-pharmaceutically acceptable salts may conveniently be used to prepare pharmaceutically acceptable salts thereof and are not excluded from the scope of the invention. Such pharmacologically and pharmaceutically acceptable salts include, but are not limited to, those prepared from the following acids: hydrochloric, hydrobromic, sulphuric, nitric, phosphoric, maleic, acetic, salicylic, p-toluene sulfonic, tartaric, citric, methane sulfonic, formic, malonic, succinic, naphthalene-2-sulfonic, and benzene sulfonic. Also, pharmaceutically acceptable salts can be prepared as alkaline metal or alkaline earth salts, such as sodium, potassium or calcium salts of the carboxylic acid group.

Suitable buffering agents include: acetic acid and a salt (1-2% W/V); citric acid and a salt (1-3% W/V); boric acid and a salt (0.5-2.5% W/V); and phosphoric acid and a salt (0.8-2% W/V).

Suitable preservatives include benzalkonium chloride (0.003-0.03% W/V); chlorobutanol (0.3-0.9% W/V); parabens (0.01-0.25% W/V) and thimerosal (0.004-0.02% W/V).

A variety of administration routes are available. The particular mode selected will depend, of course, upon the particular drug or combination of drugs selected, the severity of the condition being treated, the condition of the patient, and the dosage required for therapeutic efficacy. The methods of this invention, generally speaking, may be practiced using any mode of administration that is medically acceptable, meaning any mode that produces effective levels of the active compounds without causing clinically unacceptable adverse effects. Such modes of administration include oral, rectal, topical, nasal, other mucosal forms, direct injection, transdermal, sublingual or other routes. “Parenteral” routes include subcutaneous, intravenous, intramuscular, or infusion. Direct injection may be preferred for local delivery to the site of fibril aggregation. Oral administration may be preferred for prophylactic treatment e.g., in a subject at risk of developing a disease associated with fibril formation, because of the convenience to the patient as well as the dosing schedule.

Chemical/physical vectors may be used to deliver the agents of the invention to a target (e.g. cell) and facilitate uptake thereby. As used herein, a “chemical/physical vector” refers to a natural or synthetic molecule, other than those derived from bacteriological or viral sources, capable of delivering the agent of the invention to a target (e.g. cell).

A preferred chemical/physical vector of the invention is a colloidal dispersion system. Colloidal dispersion systems include lipid-based systems including oil-in-water emulsions, micelles, mixed micelles, and liposomes. A preferred colloidal system of the invention is a liposome. Liposomes are artificial membrane vessels which are useful as a delivery vector in vivo or in vitro. It has been shown that large unilamellar vessels (LUV), which range in size from 0.2-4.0.mu. can encapsulate large macromolecules. RNA, DNA, and intact virions can be encapsulated within the aqueous interior and be delivered to cells in a biologically active form (Fraley, et al., Trends Biochem. Sci., v. 6, p. 77 (1981)). In order for a liposome to be an efficient gene transfer vector, one or more of the following characteristics should be present: (1) encapsulation of the gene of interest at high efficiency with retention of biological activity; (2) preferential and substantial binding to a target cell in comparison to non-target cells; (3) delivery of the aqueous contents of the vesicle to the target cell cytoplasm at high efficiency; and (4) accurate and effective expression of genetic information.

Liposomes may be targeted to a particular (e.g. tissue), such as (e.g. the vascular cell wall), by coupling the liposome to a specific ligand such as a monoclonal antibody, sugar, glycolipid, or protein.

Liposomes are commercially available from Gibco BRL, for example, as LIPOFECTIN™. and LIPOFECTACE™., which are formed of cationic lipids such as N-[1-(2,3 dioleyloxy)-propyl]-N,N, N-trimethylammonium chloride (DOTMA) and dimethyl dioctadecylammonium bromide (DDAB). Methods for making liposomes are well known in the art and have been described in many publications. Liposomes also have been reviewed by Gregoriadis, G. in Trends in Biotechnology, V. 3, p. 235-241 (1985).

In one particular embodiment, the preferred vehicle is a biocompatible micro particle or implant that is suitable for implantation into the mammalian recipient. Exemplary bioerodible implants that are useful in accordance with this method are described in PCT International application no. PCT/US/03307 (Publication No. WO 95/24929, entitled “Polymeric Gene Delivery System”, claiming priority to U.S. patent application Ser. No. 213,668, filed Mar. 15, 1994). PCT/US/0307 describes a biocompatible, preferably biodegradable polymeric matrix for containing an exogenous gene under the control of an appropriate promoter. The polymeric matrix is used to achieve sustained release of the exogenous gene in the patient. In accordance with the instant invention, the agent of the invention is encapsulated or dispersed within the biocompatible, preferably biodegradable polymeric matrix disclosed in PCT/US/03307. The polymeric matrix preferably is in the form of a micro particle such as a micro sphere (wherein the agent is dispersed throughout a solid polymeric matrix) or a microcapsule (wherein the agent is stored in the core of a polymeric shell). Other forms of the polymeric matrix for containing the agents of the invention include films, coatings, gels, implants, and stents. The size and composition of the polymeric matrix device is selected to result in favorable release kinetics in the tissue into which the matrix device is implanted. The size of the polymeric matrix devise further is selected according to the method of delivery which is to be used, typically injection into a tissue or administration of a suspension by aerosol into the nasal and/or pulmonary areas. The polymeric matrix composition can be selected to have both favorable degradation rates and also to be formed of a material which is bioadhesive, to further increase the effectiveness of transfer when the devise is administered to a vascular surface. The matrix composition also can be selected not to degrade, but rather, to release by diffusion over an extended period of time.

Both non-biodegradable and biodegradable polymeric matrices can be used to deliver agents of the invention of the invention to the subject. Biodegradable matrices are preferred. Such polymers may be natural or synthetic polymers. Synthetic polymers arc preferred. The polymer is selected based on the period of time over which release is desired, generally in the order of a few hours to a year or longer. Typically, release over a period ranging from between a few hours and three to twelve months is most desirable. The polymer optionally is in the form of a hydrogel that can absorb up to about 90% of its weight in water and further, optionally is cross-linked with multi-valent ions or other polymers.

In general, the agents of the invention are delivered using the bioerodible implant by way of diffusion, or more preferably, by degradation of the polymeric matrix. Exemplary synthetic polymers which can be used to form the biodegradable delivery system include: polyamides, polycarbonates, polyalkylenes, polyalkylene glycols, polyalkylene oxides, polyalkylene terepthalates, polyvinyl alcohols, polyvinyl ethers, polyvinyl esters, polyvinyl halides, polyvinylpyrrolidone, polyglycolides, polysiloxanes, polyurethanes and co-polymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), polyethylene, polypropylene, poly(ethylene glycol), poly(ethylene oxide), poly(ethylene terephthalate), poly(vinyl alcohols), polyvinyl acetate, poly vinyl chloride, polystyrene and polyvinylpyrrolidone.

Examples of non-biodegradable polymers include ethylene vinyl acetate, poly(meth)acrylic acid, polyamides, copolymers and mixtures thereof.

Examples of biodegradable polymers include synthetic polymers such as polymers of lactic acid and glycolic acid, polyanhydrides, poly(ortho)esters, polyurethanes, poly(butic acid), poly(valeric acid), and poly(lactide-cocaprolactone), and natural polymers such as alginate and other polysaccharides including dextran and cellulose, collagen, chemical derivatives thereof (substitutions, additions of chemical groups, for example, alkyl, alkylene, hydroxylations, oxidations, and other modifications routinely made by those skilled in the art), albumin and other hydrophilic proteins, zein and other prolamines and hydrophobic proteins, copolymers and mixtures thereof. In general, these materials degrade either by enzymatic hydrolysis or exposure to water in vivo, by surface or bulk erosion.

Bioadhesive polymers of particular interest include bioerodible hydrogels described by H. S. Sawhney, C. P. Pathak and J. A. Hubell in Macromolecules, 1993, 26, 581-587, the teachings of which are incorporated herein, polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate). Thus, the invention provides a composition of the above-described agents for use as a medicament, methods for preparing the medicament and methods for the sustained release of the medicament in vivo.

The compositions may conveniently be presented in unit dosage form and may be prepared by any of the methods well known in the art of pharmacy. All methods include the step of bringing the therapeutic agents into association with a carrier which constitutes one or more accessory ingredients. In general, the compositions are prepared by uniformly and intimately bringing the therapeutic agent into association with a liquid carrier, a finely divided solid carrier, or both, and then, if necessary, shaping the product.

Compositions suitable for parenteral administration conveniently comprise a sterile aqueous preparation of the therapeutic agent, which is preferably isotonic with the blood of the recipient. This aqueous preparation may be formulated according to known methods using those suitable dispersing or wetting agents and suspending agents. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono or di-glycerides. In addition, fatty acids such as oleic acid find use in the preparation of injectables. Carrier formulations suitable for oral, subcutaneous, intravenous, intramuscular, etc. can be found in Remington's Pharmaceutical Sciences, Mack Publishing Company, Easton, Pa.

Compositions suitable for oral administration may be presented as discrete units such as capsules, cachets, tablets, or lozenges, each containing a predetermined amount of the therapeutic agent. Other compositions include suspensions in aqueous liquors or non-aqueous liquids such as a syrup, an elixir, or an emulsion.

Other delivery systems can include time-release, delayed release or sustained release delivery systems. Such systems can avoid repeated administrations of the therapeutic agent of the invention, increasing convenience to the subject and the physician. Many types of release delivery systems are available and known to those of ordinary skill in the art. They include polymer based systems such as polylactic and polyglycolic acid, poly(lactide-glycolide), copolyoxalates, polyanhydrides, polyesteramides, polyorthoesters, polyhydroxybutyric acid, and polycaprolactone. Microcapsules of the foregoing polymers containing drugs are described in, for example, U.S. Pat. No. 5,075,109. Nonpolymer systems that are lipids including sterols such as cholesterol, cholesterol esters and fatty acids or neutral fats such as mono-, di- and tri-glycerides; liposomes; phospholipids; hydrogel release systems; silastic systems; peptide based systems; wax coatings, compressed tablets using conventional binders and excipients, partially fused implants and the like. Specific examples include, but are not limited to: (a) erosional systems in which the polysaccharide is contained in a form within a matrix, found in U.S. Pat. Nos. 4,452,775, 4,675,189, and 5,736,152, and (b) diffusional systems in which an active component permeates at a controlled rate from a polymer such as described in U.S. Pat. Nos. 3,854,480, 5,133,974 and 5,407,686. In addition, pump-based hardware delivery systems can be used, some of which are adapted for implantation.

Use of a long-term sustained release implant may be particularly suitable for treatment of established conditions as well as subjects at risk of developing a disease associated with fibril formation. “Long-term” release, as used herein, means that the implant is constructed and arranged to deliver therapeutic levels of the active ingredient for at least 7 days, and preferably 30-60 days. The implant may be positioned at the site of injury. Long-term sustained release implants are well known to those of ordinary skill in the art and include some of the release systems described above.

According to certain methods of the invention the compositions can be administered by injection by gradual infusion over time or by any other medically acceptable mode. The administration may, for example, be intravenous, intraperitoneal, intramuscular, intracavity, subcutaneous or transdermal. Preparations for parenteral administration includes sterile aqueous or nonaqueous solutions, suspensions and emulsions. Examples of nonaqueous solvents are propylene glycol, polyethylene glycol, vegetable oil such as olive oil, an injectable organic esters such as ethyloliate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Intravenous vehicles include fluid and nutrient replenishers, electrolyte replenishers, (such as those based on Ringer's dextrose), and the like. Preservatives and other additives may also be present such as, for example, antimicrobials, antioxidants, chelating agents, and inert gases and the like. Those of skill in the art can readily determine the various parameters for preparing these alternative pharmaceutical compositions without resort to undue experimentation.

The compositions may conveniently be presented in unit dosage form and may be prepared by any of the methods well known in the art of pharmacy. In general, the compositions are prepared by uniformly and intimately bringing the active compounds into association with a liquid carrier, a finely divided solid carrier, or both, and then, if necessary, shaping the product.

Compositions suitable for oral administration may be presented as discrete units such as capsules, cachettes, tablets, or lozenges, each containing a predetermined amount of the active compound. Other compositions include suspensions in aqueous liquors or non-aqueous liquids such as a syrup, an elixir, or an emulsion.

Other delivery systems can include time-release, delayed release or sustained release delivery systems. Such systems can avoid repeated administrations of the active compounds of the invention, increasing convenience to the subject and the physician. Many types of release delivery systems are available and known to those of ordinary skill in the art. They include polymer based systems such as polylactic and polyglycolic acid, polyanhydrides and polycaprolactone; nonpolymer systems that are lipids including sterols such as cholesterol, cholesterol esters and fatty acids or neutral fats such as mono-, di and triglycerides; hydrogel release systems; silastic systems; peptide based systems; wax coatings, compressed tablets using conventional binders and excipients, partially fused implants and the like. Specific examples include, but are not limited to: (a) erosional systems in which the polysaccharide is contained in a form within a matrix, found in U.S. Pat. Nos. 4,452,775 (Kent); 4,667,014 (Nestor et al.); and 4,748,034 and 5,239,660 (Leonard) and (b) diffusional systems in which an active component permeates at a controlled rate through a polymer, found in U.S. Pat. Nos. 3,832,253 (Higuchi et al.) and 3,854,480 (Zaffaroni). In addition, a pump-based hardware delivery system can be used, some of which are adapted for implantation.

Use of a long-term sustained release implant may be particularly suitable in some cases. “Long-term” release, as used herein, means that the implant is constructed and arranged to deliver therapeutic levels of the active ingredient for at least 30 days, and preferably 60 days. Long-term sustained release implants are well known to those of ordinary skill in the art and include some of the release systems described above.

### EXAMPLES

Colloid Preparation

Colloids were prepared for drug screening in a manner similar to that described in International Patent Publication No. WO 00/43791, published Jul. 27, 2000, entitled “Rapid and Sensitive Detection of Aberrant Protein Aggregation in Neurodegenerative Diseases”, incorporated herein by reference.

In a typical example, 1.5 ml of commercially available gold colloid (Auro Dye by Amersham) were pelleted by centrifugation in a microfuge on high for 10 minutes. The pellet was resuspended in 100° L of the storage buffer (sodium citrate and tween-20). 100° L of a dimethyl formamide (DMF) solution containing thiols. Following a 3-hour incubation in the thiol solution, the colloids were pelleted and the supernatant discarded. They were then heat cycled in 100° L of 400° M tri-ethylene glycol-terminated thiol in DMF for 2 minutes at 55° C., 2 minutes at 37° C., 1 minute at 55° C., 2 minutes at 37° C., then room temperature for 10 minutes. Heat cycling results in the elimination of any species that are not in the lowest energy confirmation, resulting in a stable, close-packed, self-assembled monolayer. Heat cycling can be carried out with any of a wide variety of self-assembled monolayer-forming species. The colloids were then pelleted and 100° L 100 mM NaCl phosphate buffer were added. The colloids were then diluted 1:1 with 180 M NiSO₄ in the colloid storage buffer.

Thiols used in coating colloids typically were derived from solutions containing about 40° M nitrilo tri-acetic acid (NTA)-thiol, and other thiols such as methyl-terminated thiol (HS—(CH2)15 CH3), 40% tri-ethylene glycol-terminated thiol, HS(CH₂)₁₁(CH₂CH2)₃OH, (formula) and 50% poly (ethynylphenyl) thiol (C₁₆H₁₀S). Different thiols were used to selectively inhibit non-specific binding optimally.

In this and certain other embodiments of the invention, use is made of self-assembled monolayers (SAMs) on surfaces of colloid particles. In one set of preferred embodiments, SAMs formed completely of synthetic molecules completely cover a surface or a region of a surface, e.g. completely cover the surface of a colloid particle. “Synthetic molecule”, in this context, means a molecule that is not naturally occurring, rather, one synthesized under the direction of human or human-created or human-directed control. “Completely cover” in this context, means that there is no portion of the surface or region that directly contacts a protein, antibody, or other species that prevents complete, direct coverage with the SAM. I.e. in preferred embodiments the surface or region includes, across its entirety, a SAM consisting completely of non-naturally-occurring molecules (i.e. synthetic molecules). The SAM can be made up completely of SAM-forming species that form close-packed SAMs at surfaces, or these species in combination with molecular wires or other species able to promote electronic communication through the SAM (including defect-promoting species able to participate in a SAM), or other species able to participate in a SAM, and any combination of these. Preferably, all of the species that participate in the SAM include a functionality that binds, optionally covalently, to the surface, such as a thiol which will bind to a gold surface covalently.

A self-assembled monolayer on a surface, in accordance with an embodiments of the invention, can be comprised of a mixture of species (e.g. thiol species when gold is the surface) that can present (expose) essentially any chemical or biological functionality. For example, they can include tri-ethylene glycol-terminated species (e.g. tri-ethylene glycol-terminated thiols) to resist non-specific adsorption, and other species (e.g. thiols) terminating in a binding partner of an affinity tag, e.g. terminating in a chelate that can coordinate a metal such as nitrilotriacetic acid which, when in complex with nickel atoms, captures histidine-tagged binding species. An embodiment of the present invention provides a method for rigorously controlling the concentration of essentially any chemical or biological species presented on a colloid surface, for example for assays informative of conditions relating to neurodegenerative disease and/or treatment thereof. In many embodiments of the invention the self-assembled monolayer is formed on gold colloid particles.

Pre-formed aggregates, characteristic of neurodegenerative disease and other diseases characterized by aberrant protein aggregation, and other non-disease processes, can be sensitively detected by monitoring color change and visible fibril formation as a function of time. Detection of aggregate formation or lack thereof, in the presence of candidate drugs, was conducted as follows.

Gold colloids were derivatized with nitrilo tri-acetic acid/nickel self-assembled monolayers (NTA-Ni-SAMs), for the capture of histidine-tagged proteins. Colloids were coated with a low density of NTA-Ni (40° M NTA-thiol in a total thiol concentration of 1000:M) to inhibit the aggregation of nearest neighbor peptides, immobilized on a common colloidal particle. 30° L aliquots of the derivatized colloids were placed in wells of a 96-well plate. Histidine-tagged·-amyloid peptides (amino acids 1-40) were dissolved in phosphate buffer pH 7.4, then added to the colloid solutions such that the final concentration in a 100° L volume was 14° M. Pre-formed fibrils, made from synthetic non-histidine-tagged·-amyloid peptides, were serially diluted and added to the solutions such that the final concentration of fibrils varied from 330 nanomolar to 50 picomolar. For every assay (each well), a negative control assay, in which no fibril was added, was performed. Solutions were incubated at 37° C. for 1 hour and care was taken not to agitate the solutions. The color of the solutions that contained fibrils, changed from the characteristic pink color of gold colloids, to a deep purple/gray. Solutions that contained hisitidine-tagged·-amyloid (1-40), but no pre-formed fibrils changed to a lighter purple color, and at a slower rate than solutions to which fibrils were added. Dark masses, in the centers of wells that contained pre-formed fibrils, were clearly visible with the unaided eye. Observation using 40-fold magnification with a dissecting microscope revealed large, purple reticulums. These aggregates were clearly suspended in the fluid medium rather than sedimented onto the bottom of the plate. Negative control wells showed a grainy precipitate, but no large aggregates. Visual examination allowed us to discriminate between wells that contained pre-formed fibril and those that did not, down to an added fibril concentration of 50 picomolar. Results were recorded by photographs taken with a Nikon camera (ASA 800 film) attached to an inverted microscope at 40-fold magnification. At higher peptide and fibril concentration, large colloid-decorated fibril structures are visible.

Inhibitory drugs may act on Alzheimer's disease β-amyloid peptides either by binding to normal peptides in a way that inhibits conversion to the fibrillar beta-sheet form, or by binding to existing fibrillar aggregates in a way that prevents further conversion of normal AB to the fibrillar form. In identifying compositions suitable for treatment of neurodegenerative disease according to some embodiments of the invention, drug candidates were screened for Alzheimer's Disease inhibition in two different ways.

In one drug screening method, histidine-tagged AB 1-40 peptides were immobilized on colloids derivatized with self-assembled monolayers containing NTA-Ni for the attachment of histidine-tagged peptides. It has been previously shown that the Aβ peptides, if not inhibited by a drug, will spontaneously convert to the fibrillar form and aggregate, pulling the colloids close together, and causing a color change from pink to blue.

Drugs from the Sigma-RBI Library of Pharmacologically Active Compounds (LOPAC; #SC001, 1999) were dissolved in dimethyl sulphoxide (DMSO) to a final concentration of 2.9 mg/ml. 5 ul of each dissolved drug was added to individual wells of a 96-well ELISA plate, which also contained 55 ul of phosphate buffer (10 mM phosphate, pH 7.4, 100 mM sodium chloride). 10 ul of a 50 uM solution of his-Aβ 1-40 peptide in phosphate buffer was added to each well, for a final concentration of 5 uM in a final volume of 100 ul. 30 ul of colloids derivatized with SAMs containing 40 uM NTA-Ni was then added to each well, for a final volume of 10 ul. In each drug-screening plate, four negative control wells were run that contained DMSO in place of a drug candidate, and a random histidine-tagged peptide sequence in place of Aβ 1-40. Four wells of a positive control were also run, which contained only DMSO in place of a drug candidate, to show when the Aβ would aggregate in the absence of any aggregation-accelerating or inhibiting drugs. The drug screening plates were then incubated at 37C for a period of between 1.5-2 hours, or until the positive control wells turned blue, indicating that aggregation had occurred. The plates were then photographed on a CCD camera. Drugs that inhibited fibril formation remained pink when the positive controls turned blue (a color change from pink to blue indicates that the drug candidate had no effect on fibril formation). Plates were then incubated for a longer period of time to assess for how long the inhibitory drug would work.

In another drug screening method, drugs were incubated with Aβ 1-42 fibrils, which will convert normal Aβ 1-40 peptides to the fibrillar form at a faster rate than Aβ 1-40 fibrils. Histidine-tagged Aβ 1-40 peptides were then added to the wells, along with colloids derivatized with self-assembled monolayers containing NTA-Ni for the attachment of histidine-tagged Aβ 1-40. It was previously shown that Aβ 1-42 fibrils will cause accelerated conversion of Aβ 1-40, which will then incorporate into large aggregates structures. These aggregates are colored red by the colloids attached to the peptides, and are easily visible under the microscope.

5 ul of each drug in DMSO was added to the wells of a 96-well ELISA plate, which contained 52 ul of phosphate buffer. 3.3 ul of 30 uM Aβ 1-42 fibrils (non-histidine-tagged) was added to each well, for a final concentration of 1 uM in a final volume of 100 ul. 10 ul of a 200 uM solution of his-Aβ 1-40 peptide in phosphate buffer was added to each well, for a final concentration of 20 uM in a final volume of 100 ul. 30 ul of colloids derivatized with SAMs containing 40 uM NTA-Ni was then added to each well, for a final volume of 100 ul. In each drug-screening plate, four negative control wells were run that contained DMSO in place of a drug candidate, and a random histidine-tagged peptide sequence in place of Aβ 1-40. Four wells of a positive control were also run, which contained only DMSO in place of a drug candidate, to show when the Aβ would aggregate in the absence of any aggregation-accelerating or inhibiting drugs. Plates were incubated at 37° C. for 4-6 hours, or until large structures were visible under the microscope at 40× magnification. Drugs that inhibited peptide conversion or incorporation into aggregate structures showed a visibly lesser degree of aggregation than the positive controls. Each well of the ELISA plate was given a value of 1-3”+” signs as a measure of aggregation inhibition, “0” if the drug had no effect on aggregation, or 1-3“−” signs as a measure of aggregation acceleration.

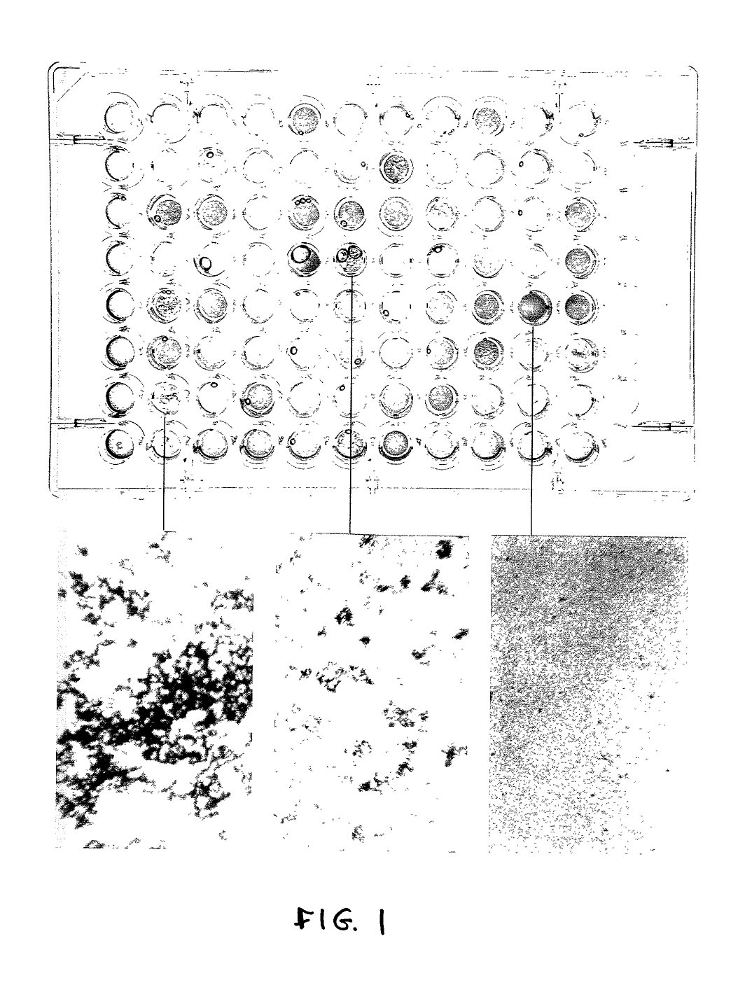

Rack 4 of the Sigma-RBI Library of Pharmacologically Active Compounds (LOPAC #SC001; 1999; Sigma) was screened for inhibition of fibril formation at 5 uM His-Aβconcentration. 5 ul of drug was added to the wells of a 96-well ELISA plate, which contained 55 ul phosphate buffer, pH 7.4. 10 ul of 50 uM His-Aβ was added per well, for a final concentration of 5 uM. 30 ul of SAM-derivitized colloids was then added per well to surface-immobilize the histidine-tagged peptides. Plates were incubated at 37° C. for approximately two hours to allow peptide-aggregation to occur, and then photographed on a CCD camera. FIG. 1 is a photograph of the rack 4 drug screen after two hour of incubation. Wells A-D in column 1 are the positive controls, to which no drug was added. The blue color shows that peptide aggregation occurred, pulling the colloids into close proximity, and thus causing the color change from pink to blue. Wells E-H in row 1 are the negative controls, containing an irrelevant peptide sequence that does not aggregate in place of Aβ peptide. The pink color of these wells shows that no aggregation occurred, and the colloids are still spaced far apart in solution. Well E4 is an example of a drug that had no effect on fibril formation, as the color of the solution quickly turned blue. Well E10 is an example of a drug that inhibits fibril formation, as the solution remained pink even after several hours of incubation.

In FIG. 1, initial color change was recorded using a digital camera. The aggregation was allowed to proceed at 37° C. for two more hours. Each well was then photographed individually under 40-fold magnification. The magnification of wells G2 (blue), D6 (blue) and E10 (pink) are shown as indicated.

Monitoring Drug Activity as a Function of Time for Drug Profiling

To determine at what stage of aggregate formation a particular drug is effective, for how long the drug is effective, and whether the drug is able to reserve aggregate formation, a drug profiling experiment was run in which many points were taken over time using a 96-well plate spectrophotometer.

To each well of a 96-well plate, 55 ul phosphate buffer (pH 7.4), 30 ul NTA-SAM derivitized colloids, 10 ul, 50 um His-AB, and 5 ul drug candidate or DMSO (for control wells) were added. Negative control wells contained 10 ul, 50 um irrelevant Histidine-tagged peptide in place of His-AB, and DMSO in place of a drug candidate. Positive control wells did contain His-AB, but contained DMSO in place of a drug candidate. Absorbance at 530 nm was read every 5 minutes for 2 hours in a soft-max Pro 96-well spectrophotometer (molecular devices), while an identical 96-well plate was observed and photographed on the benchtop.

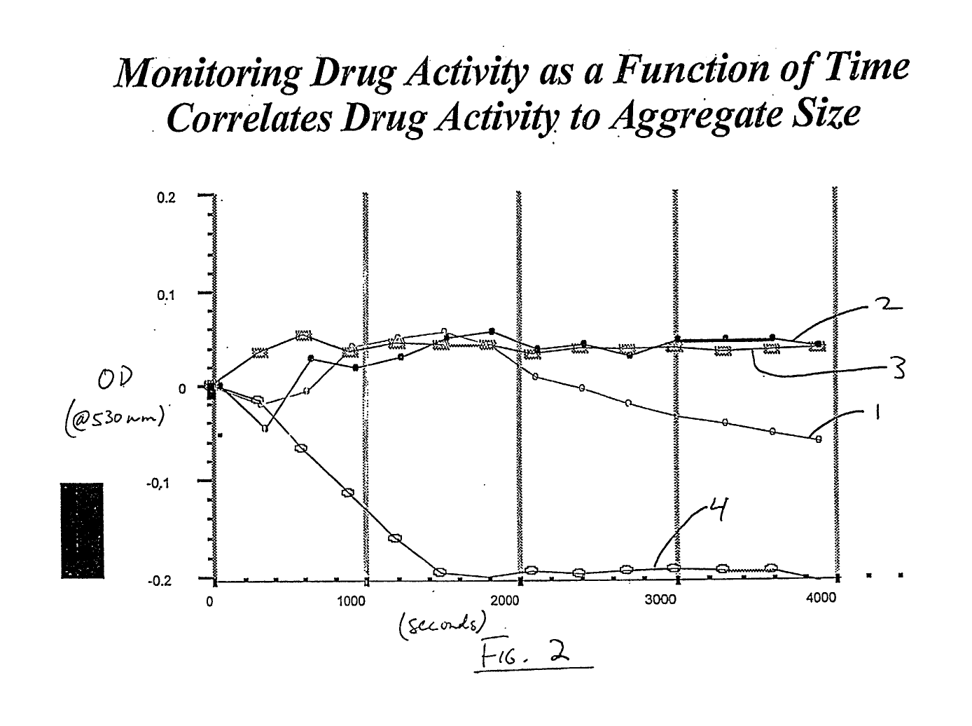

Negative control wells remained red, and positive control wells turned blue within 1 hour. Wells containing drug candidates varied in color over time depending on the effectiveness of the drug candidate. Absorbance at 530 nm was plotted against time for each well. Wells that turned blue, indicating no drug or no drug activity, showed a significant drop in absorption at 530 over time. Wells that contained drugs which inhibited aggregation showed a constant absorbance at 530 nm over the range of aggregate size on which the drug was effective, the drug was effective. In this manner, drug efficiency was determined. FIG. 2 shows the drug profiles of two drugs that inhibited aggregation (1 and 2) plotted with a positive control (3) and a negative control (4). It can be seen that the two drugs work on different size aggregates for different periods of time.

The results of this experiment demonstrate the strength of another aspect of the invention, which involves determining an appropriate drug for treatment of a physiological process involving aggregation at a particular stage of the process or under conditions of particular aggregate size, or both. Referring to FIG. 2, it can be seen that the drug represented by curve 1 shows greater effectiveness at an earlier stage of aggregate formation and/or with respect to smaller aggregates, and the drug represented by curve two shows greater effectiveness at a later stage of aggregate formation and/or with respect to larger aggregates. Conducting experiments such as this with a variety of candidate drugs can provide information for a treatment protocol based upon the stage of a physiological process and/or aggregate size in a patient.

Cell-Based Screening Assay for Candidate Drugs for Affecting Aggregate Formation at a Variety of Stages of Biochemical Progression

In this example, it is described how one tests a candidate drug for activity in affecting cellular processes involved in aggregation-associated disease including neurodegenerative disease and non-neurodegenerative disease, and non-disease processes. A whole cell assay was designed to enable the screening of drugs for their ability to affect, directly or indirectly, beta-amyloid production, processing including cleavage, and secretion (Wolfe, et al., Nature, 1999 April 8; 398 (6727):513-7; Haass, Nat. Med. December, 1999; 1 (12):1291-6; LaBlanc, et al., J Neurosci Res., April, 1992, 31(4):635-45).

Amyloid precursor protein (APP: the protein precursor of beta-amyloid) was stably transfected into human embryonic kidney cells. Selkoe, “The Cell Biology of Beta-Amyloid Precursor Protein and Presenilin in Alzheimer's Disease.”, Trends in Cell Biology, 8:11, 447-453, November, 1998; Knops, et al. “Protein-type Specific-Inhibition of A-Beta Release By Bafilomycin A1, A Selective Inhibitor of Vaculor ATP ases”, Journal of Biological Chemistry, 270:6, 2419-2422, Feb. 10, 1995). Cells (100 ul vol at 40% confluency) were dispensed into a 96-well plate and grown in DMEM (Dulbecco modified eagle medium) in a CO₂ incubator at 37 degrees C. for 21 hrs. Colloids (30 ul) derivatized with NTA-SAMs (4% NTA in the thiol solution) were mixed with histidine-tagged beta-amyloid peptide (1-40) then added to each well of the micro titer plate. The final beta-amyloid concentration in each well was 17 uM. Control wells contained either: 1) histidine-tagged GST instead of beta-amyloid peptide; or 2) everything as above except no cells were present in the growth media; or 3) cells that were not transfected with APP.

When colloids and peptides were added at 21 hrs., only solutions containing cells expressing the APP plasmid, colloids and His-tagged beta-amyloid turned from the characteristic pink to purple. Solutions in the control wells remained pink.

Another batch of cells treated as described above, were allowed to grow for an additional 18 hrs. after colloids and beta-amyloid were added. These cells continued to extrude APP products (beta-amyloid 1-40 and 1-42). When observed under 40-fold magnification, a halo of accumulated colloid-peptide aggregates could be seen around each cell. Cells incubated with colloids and control his-tagged peptides showed no regional accumulation of colloids. Control cells (no APP transfection) incubated with beta-amyloid and colloids also showed no signs of colloid aggregation. Control solutions containing colloids, beta-amyloid and cell growth medium also did not show signs of colloid aggregation.

This example represents yet another aspect of the invention involving exposing a candidate drug for inhibition of neurodegenerative disease to a cell adapted to secrete neurodegenerative disease fibril- or aggregate-forming species, and determining the effect of the candidate drug on the aggregation potential of material secreted from the cell. This example also presents a technique for determining drug toxicity by determining the effect of the candidate drug on a cell.

Colorimetric Screening Assay for Candidate Drugs that Effect APP Production, Secretion, or Cleavage and thus Affect Amyloid Aggregation

Human Embryonic Kidney (HEK) cells expressing the Amyloid Precursor Protein (APP) were split into the wells of a 96-well cell-culture plate and were allowed to adhere and spread. Just after the cells had spread onto the plate, 1.25 ul of 2.9 mg/mL drug in DMSO was added to each well of the plate. Positive control cells were given DMSO in place of a drug. Negative controls were HEK cells that had not been transfected with the APP gene, and were given DMSO in place of a drug. Cells were allowed to grow overnight for approximately 18 hours at 37C. After 18 hours, 1.925 mL of a 30 uM solution of His-tagged AB(1-40) in phosphate buffer was mixed with 2.880 mL 40 uM NTA colloids for a resulting concentration of 12 uM His-AB. 50 ul of the AB colloid solution was added to each well of the cell-culture plate, which contained 70 ul cell culture media per well. This resulted in a final volume of 120 ul per well, with a concentration of 5 uM His-AB per well. The plates were incubated at 37C for 8-12 hours, or until a color change from pink to blue occurred in the positive controls.

Once the positive controls changed color from pink to blue, the wells containing drug candidates were visually examined for a color change. A color change from pink to blue indicates that the drug candidate did not affect production, secretion, or cleavage of the APP protein. A lack of color change (those wells that remain pink) indicates that the APP protein was effected by the drug in one of the aforementioned ways. Cell death due to drug toxicity would obviously effect APP production, and may cause a well to remain pink. For this reason, all wells containing drug candidates were examined under the microscope for cell viability. Several drugs were identified using this assay that effect APP production, secretion, or cleavage, without killing the cells, and structures of these drugs are shown in FIGS. 3 and 4.

Those skilled in the art would readily appreciate that all parameters listed herein are meant to be exemplary and that actual parameters will depend upon the specific application for which the methods and apparatus of the present invention are used. It is, therefore, to be understood that the foregoing embodiments are presented by way of example only and that, within the scope of the appended claims and equivalents thereto, the invention may be practiced otherwise than as specifically described. In the claims, all transitional phrases such as “comprising”, “including”, “carrying”, “having”, “containing”, “involving”, and the like are to be understood to be open-ended, i.e. to mean including but not limited to. Only the transitional phrases “consisting of” and “consisting essentially of”, respectively, shall be closed or semi-closed transitional phrases as set forth in the United States Patent Office Manual of Patent Examining Procedures, section 2111.03.

What is claimed is:

1. A method comprising treating a human patient susceptible to or exhibiting symptoms of conditions characterized by aggregate-forming species associated with a disease, by administering to the patient a therapeutically effective amount of a composition having a high net polarization and comprising a structure selected from the group consisting of: 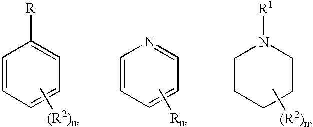
a five membered ring including at least one heteroatom, at least two rings bridged by at least one atom, at least two rings bonded directly to each other, a ring containing at least one carbonyl, and an alkyl chain;
wherein R and R² can be the same or different and each is a substituent of an organic group or chlorine, n is an integer greater than 0 and no more than 6 and each R can be the same or different and each R² can be the same or different; R¹ is a substituent of an organic group or hydrogen; any ring structure optionally including at least one carbon-carbon double bond; any ring structure optionally including at least one heteroatom selected from the group consisting of a carbonyl, nitrogen, oxygen and sulfur; each substituent being arranged so as to create polarity in the structure;
the patient being otherwise free of indication for treatment with the composition.

2. The method of claim 1, wherein the composition comprises an aromatic ring.

3. The method of claim 1, wherein the patient is susceptible to but does not exhibit symptoms of the conditions.

4. The method of claim 1, wherein the patient exhibits symptoms of the conditions.

5. The method of claim 1, wherein the disease is associated with fibril formation or aberrant protein aggregation.

6. The method of claim 5, wherein the disease is a neurodegenerative disease.

7. The method of claim 6, wherein the disease is selected from the group consisting of Familial British Dimentia, Parkinson's Disease, Alzheimer's Disease, Finnish-type Familial Amyloidoses, Huntington's Disease, AD, Frontotemporal Dementia, Senile Systemic Amyloidosis, Familial Amyloid Polyneuropathy, Transmissible Spongiform Encephalopathie, Gertsmann-Strausseler-Scheinker Syndrome, Fatal Familial Insomnia, Huntington's Chorea, Kuru, Familial amyloid polyneuropathy, Creutzfeldt Jakob, Scrapie, and Bovine Spongiform Encephalopathy.

8. The method of claim 1, wherein the composition is capable of crossing the blood/brain barrier.

9. The method of claim 5, wherein the disease is a non-neurodegenerative disease.

10. The method of claim 9, wherein the disease is selected from the group consisting of multiple myeloma, Waldenstroms Macroglobulinemia, blockage of affected blood vessels caused by precipitates of cryoglobulins, Disseminated intravascular coagulation, Glanzmann's thrombasthenia, Abnormal fibronectin aggregation, Sickle cell anemia, stroke and Type II Diabetes.

11. The method of claim 1, wherein the composition is selected from the group consisting of DNA bases, RNA bases, DNA base analogs, RNA base analogs, DNA base derivatives and RNA base derivatives.

12. The method of claim 11, wherein the composition is an anti-metabolite.

13. The method of claim 12, wherein the composition is a chemotherapy agent.

14. A method as in claim 1, wherein the structure is not:
R1—R2
wherein R1 and R2 can be the same or different and each is able to bind a neurodegenerative disease aggregate-forming species or aggregate, and (—) is a tether having length and flexibility sufficient such that R1 and R2 each are able to bind a different neurodegenerative disease aggregate forming species or aggregate.

15. A method as in claim 1, wherein the patient is free of indication for treatment for central nervous system neuronal damage, as caused by aggregate formation.

16. A method as in claim 15, comprising administering the composition to a patient free of symptoms of dementia.

17. A method as in claim 1, comprising administering to the patient the composition demonstrates the ability to inhibit conversion of peptides related to a pathogenic state of neurodegenerative disease from a soluble, monomeric state to an insoluble, beta-sheet, oligomeric state characteristic of abnormal protein deposits found in the human brain in connection with neurodegenerative disease.

18. The method of claim 1, wherein the composition demonstrates the ability to inhibit conversion of peptides related to a pathogenic state of neurodegenerative disease from a α-helix to an insoluble, beta-sheet, oligomeric state characteristic of abnormal protein deposits found in the human brain in connection with neurodegenerative disease.

19. The method of claim 1, wherein the composition demonstrates the ability to inhibit conversion of peptides related to a pathogenic state of neurodegenerative disease from a soluble, monomeric state to an early intermediate aggregate.

20. The method of claim 1, comprising treating the patient with at least two different compositions effective against at least two different stages of the condition.

21. The method of claim 1, further comprising treating the patient with a dose of normal DNA or RNA bases.

22. A method comprising:
promoting the prevention or treatment of a disease caused by aggregate formation via administration of a composition having a high net polarization and comprising a structure selected from the group consisting of: 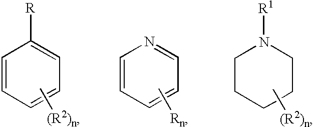
a five membered ring including at least one heteroatom, at least two rings bridged by at least one atom, at least two rings bonded directly to each other, a ring containing at least one carbonyl, and an alkyl chain;
wherein R and R² can be the same or different and each is a substituent of an organic group or chlorine, n is an integer greater than 0 and no more than 6 and each R can be the same or different and each R² can be the same or different; R¹ is a substituent of an organic group or hydrogen; any ring structure optionally including at least one carbon-carbon double bond; any ring structure optionally including at least one heteroatom selected from the group consisting of a carbonyl, nitrogen, oxygen and sulfur; each substituent being arranged so as to create polarity in the structure.

23. The method of claim 22, wherein the disease is characterized by aggregate formation.

24. A method of treatment comprising:
treating the patient with a composition having a high net polarization and comprising a structure selected from the group consisting of: 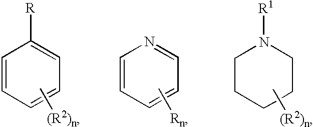
a five membered ring including at least one heteroatom, at least two rings bridged by at least one atom, at least two rings bonded directly to each other, a ring containing at least one carbonyl, and an alkyl chain;
wherein R and R² can be the same or different and each is a substituent of an organic group or chlorine, n is an integer greater than 0 and no more than 6 and each R can be the same or different and each R² can be the same or different; R¹ is a substituent of an organic group or hydrogen; any ring structure optionally including at least one carbon-carbon double bond; any ring structure optionally including at least one heteroatom selected from the group consisting of a carbonyl, nitrogen, oxygen and sulfur; each substituent being arranged so as to create polarity in the structure;
drawing a sample from the patient; and
monitoring the sample over at least two different points in time.

25. The method of claim 24, wherein the step of monitoring involves subjecting the sample to an assay.

26. The method of claim 25, wherein the assay comprises adding a plurality of binding species capable of binding to an aggregate.

27. The method of claim 25, wherein the step of monitoring further comprises detecting results of the assay.

28. The method of claim 27, wherein the step of detecting comprises detecting aggregate formation.

29. The method of claim 28, wherein the step of detecting occurs visually.

30. The method of claim 28, wherein the step of detecting occurs via spectroscopy.

31. A method of treatment, comprising:
withdrawing a sample from the patient;
subjecting the sample to a composition having a high net polarization and comprising a structure selected from the group consisting of: 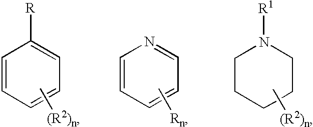
a five membered ring including at least one heteroatom, at least two rings bridged by at least one atom, at least two rings bonded directly to each other, a ring containing at least one carbonyl, and an alkyl chain;
wherein R and R² can be the same or different and each is a substituent of an organic group or chlorine, n is an integer greater than 0 and no more than 6 and each R can be the same or different and each R² can be the same or different; R¹ is a substituent of an organic group or hydrogen; any ring structure optionally including at least one carbon-carbon double bond; any ring structure optionally including at least one heteroatom selected from the group consisting of a carbonyl, nitrogen, oxygen and sulfur; each substituent being arranged so as to create polarity in the structure;
determining whether to treat the patient with the composition.

32. The method of claim 31, wherein the step of subjecting comprises adding the composition to the sample.

33. The method of claim 32, wherein the step of subjecting further comprises adding a plurality of binding species capable of binding to an aggregate.

34. The method of claim 31, wherein the step of determining comprises determining whether the composition is capable of inhibiting aggregate formation.

35. A kit comprising:
a composition having a high net polarization and comprising a structure selected from the group consisting of: 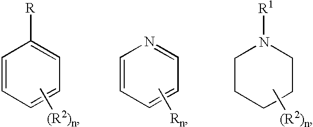
a five membered ring including at least heteroatom, at least two rings bridged by at least one atom, at least two rings bonded directly to each other, a ring containing at least one carbonyl, and an alkyl chain;
wherein R and R² can be the same or different and each is a substituent of an organic group or chlorine, n is an integer greater than 0 and no more than 6 and each R can be the same or different and each R² can be the same or different; R¹ is a substituent of an organic group or hydrogen; any ring structure optionally including at least one carbon-carbon double bond; any ring structure optionally including at least one heteroatom selected from the group consisting of a carbonyl, nitrogen, oxygen and sulfur; each substituent being arranged so as to create polarity in the structure; and
instructions for use of the composition for treatment of a disease caused by aggregate formation.

36. The kit of claim 35, wherein the disease is neurodegenerative disease.

37. The kit of claim 35, wherein the structure includes a hydrophobic portion and a hydrophilic portion.

38. The kit of claim 35, wherein the drug comprises a DNA base or base derivative.

39. The kit of claim 38, wherein the base derivative includes a nitrogen atom bonded to a sugar or a sugar derivative.

40. The kit of claim 39, wherein the sugar derivative includes a chain of at least two phosphates.

41. The kit of claim 35, wherein the drug comprises the aromatic structure: 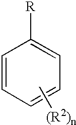
and R and R² is selected from the group consisting of a primary amine, a secondary amine, a hydroxyl, an amide, an alkylamide, a carboxyl, a carboxylic acid, a carboxylate, an ester, a sulfonate, a C₁-C₆ alkoxide optionally interrupted or terminated by a chlorine atom or a nitrogen atom and a C₁-C₆ alkyl optionally interrupted or terminated by a chlorine atom or a nitrogen atom.

42. The kit of claim 41, wherein n is zero and the drug is 1-phenylbiguanide.

43. The kit of claim 41, wherein the drug comprises the structure:

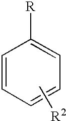

44. The kit of claim 43, wherein the drug is selected from the group consisting of phenylephrine hydrochloride and arecoline hydrobromide.

45. The kit of claim 43, wherein the drug comprises the structure: 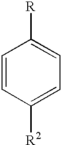
wherein R and R² are different.

46. The kit of claim 45, wherein R has an electronegativity greater than that of hydrogen, and R² has an electronegativity less than that of hydrogen.

47. The kit of claim 45, wherein the drug is selected from the group consisting of S(−)-atenolol, R(−)-atenolol, tetracaine hydrochloride, octopamine hydrochloride and procainamide hydrochloride.

48. The kit of claim 41, wherein n is at least 2 and the structure comprises at least a tri-substituted aryl ring.

49. The kit of claim 48, wherein the drug is selected from the group consisting of (±)-sulpiride, (±)-vanillyl mandelic acid, (−)-α-methyl norepinephrine, normetanephrine hydrochloride, MHPZ piperazine and erbstatin analog.

50. The kit of claim 48, wherein R² comprises a C₁-C₆ alkoxide optionally interrupted by nitrogen.

51. The kit of claim 50, wherein R comprises a C₁-C₆ alkoxide optionally interrupted by nitrogen.

52. The kit of claim 51, wherein the drug is gallamine triethiodide.

53. The kit of claim 41, wherein the aromatic structure is a first ring, R and at least one of R² is positioned on an adjacent carbon atom, the adjacent R² and R comprises a second ring fused to the first ring such that the drug comprises a fused ring structure.

54. The kit of claim 53, wherein the drug is selected from the group consisting of Metazone, ICI 11,551 hydrochloride and indatraline hydrochloride.

55. The kit of claim 53, wherein the fused ring structure comprises a tri-fused ring.

56. The kit of claim 55, wherein the drug is selected from the group consisting of (−)-physostigmine, telenzepine dihydrochloride, Loxapine succinate, Thioridazine hydrochloride, (+/−) octoclothepin maleate, luphenazine dihydrochloride and CGS-12066A dimaleate.

57. The kit of claim 55, wherein the fused ring structure comprises a tetra-fused ring.

58. The kit of claim 57, wherein the drug is selected from the group consisting of podophyllotoxin and spironolactone.

59. The kit of claim 53, wherein the second ring structure includes at least one nitrogen atom.

60. The kit of claim 59, wherein the drug is pindolol.

61. The kit of claim 35, wherein the structure is: 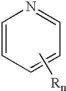
and R is selected from the group consisting of a primary amine, a secondary amine, a hydroxyl, a C₁-C₆ alkyl, a C₁-C₆ alkoxide, a carboxyl, a carboxylate, a carboxylic acid and an amide.

62. The kit of claim 61, wherein the drug is 4-aminopyridine.

63. The kit of claim 61, wherein two R substituents form a ring such that the drug comprises a fused ring structure.

64. The kit of claim 35, wherein the drug comprises the structure: 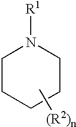
wherein the ring optionally includes a second nitrogen and optionally includes at least one carbon-carbon double bond.

65. The kit of claim 64, wherein the ring includes at least one carbonyl.

66. The kit of claim 64, wherein the drug is chlomezanone.

67. The kit of claim 64, wherein two R² groups form a ring such that the drug comprises a fused ring structure.

68. The kit of claim 67, wherein the drug is debrisoquin sulfate.

69. The kit of claim 64, wherein the ring includes two nitrogen atoms.

70. The kit of claim 69, wherein the drug is selected from the group consisting of uracil, 5-trifluoromethyl-5,6-dihydro, primidone and urapidil-5-methyl.

71. The kit of claim 69, wherein the drug comprises a fused ring structure.

72. The kit of claim 71, wherein the drug is selected from the group consisting of 8-cyclopentyl-1,3-dimethylxanthine, 1-allyl-3,7-dimethyl-8-p-sulphophenyl-xanthine, Tracazolate and alpha, beta-methylene adenosine 5′ triphosphate dilithium.

73. The kit of claim 69, wherein the drug comprises a DNA base or DNA base derivative.

74. The kit of claim 35, wherein the drug comprises a five-membered ring containing at least one heteroatom selected from the group consisting of oxygen, nitrogen, sulfur and silicon.

75. The kit of claim 74, wherein the heteroatom is nitrogen, the ring being selected from the group consisting of: 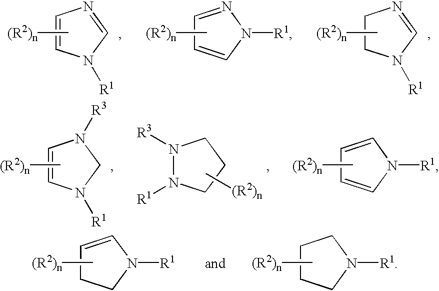
wherein R² is selected from the group consisting of an organic group and chlorine, each R² can be the same or different, n is an integer no more than 4; R¹ and R³ can be the same or different and each is selected from the group consisting of an organic group and hydrogen; any two R² groups on adjacent carbon atoms being capable of forming a ring such that the drug comprises a fused ring structure.

76. The kit of claim 75, wherein R¹ and R³ is selected from the group consisting of hydrogen, a sugar, a sugar derivative, a C₁-C₆ alkyl optionally interrupted or terminated by a nitrogen, and an aryl.

77. The kit of claim 76, wherein the drug is selected from the group consisting of histamine-R(−)-α-methyl-dihydrochloride, histamine-1-methyl-hydrochloride and cimetidine.

78. The kit of claim 76, wherein the five-membered ring includes a carbonyl.

79. The kit of claim 78, wherein the drug is selected from the group consisting of phenylbutazone and oxotremorine methiodide.

80. The kit of claim 75, wherein the five-membered ring is fused to a six-membered ring.

81. The kit of claim 80, wherein the six-membered ring is aromatic.

82. The kit of claim 81, wherein the drug is 1,3-dihydro-1-[2-hydroxy-5-(trifluoromethyl)phenyl]-5-(trifluoromethyl)-2H-benzimidazol-2-one.

83. The kit of claim 81, wherein the six-membered ring contains two nitrogen atoms.

84. The kit of claim 83, wherein the drug is N6-cyclopentyl 9-methyladenine.

85. The kit of claim 83, wherein R¹ comprises a sugar.

86. The kit of claim 85, wherein the drug is selected from the group consisting of S-(4-nitrobenzyl)-6-thioinosine, S-(4-nitrobenzyl)-6-thioguanosine, N6-methyladenosine, 2-phenylamineadenosine and alpha, beta-methylene adenosine 5′ triphosphate dilithium.

87. The kit of claim 85, wherein the sugar is bonded to a chain of at least two phosphates.

88. The kit of claim 87, wherein the drug is p,p-di(adenosine-5′)-tetraphosphate triammonium.

89. The kit of claim 83, wherein the fused rings comprise a structure selected from the group consisting of a purine and a purine derivative.

90. The kit of claim 74, wherein the heteroatom is oxygen.

91. The kit of claim 90, wherein the drug is (+)-cis-dioxolane

92. The kit of claim 35, wherein the drug comprises the structure comprising at least two rings bridged by at least one atom selected from the group consisting of carbon, nitrogen, silicon and sulfur.

93. The kit of claim 92, wherein the at least two rings are bridged by a bridging unit selected from the group consisting of a nitrogen atom, —N(R)—, —C(O)N(R)—, —N═N—, a C₁-C₆ alkyl, —C(S)N(R)—, —C(O)R— and —S(O)₂N(R)—, wherein R is selected from the group consisting of an aryl, an aryl radial, a C₁-C₆ alkyl optionally terminated by an amine, a C₁-C₆ alkyl radical and a hydrogen.

94. The kit of claim 93, wherein the drug is selected from the group consisting of diclofenac sodium, 4-[[4-formyl-5-hydroxy-6-methyl-3-[(phosphonooxy)methyl]-2-pyridinyl]azo]-1,3-benzenedisulfonic acid tetrasodium salt, phentolamine mesylate, 3,3′,4,4′-tetramethoxy-N-methyl-diphenethylamine hydrochloride, thioperamide maleate and BRL 37344 sodium.

95. The kit of claim 92, wherein the drug is selected from the group consisting of p-fluorohexahydro-sila-difenidol hydrochloride, 4-DAMP methiodide, hexahydro-sila-difenidol hydrochloride.

96. The kit of claim 35, wherein the drug comprises a structure comprising a ring containing at least one carbonyl group and containing at least one nitrogen atom.

97. The kit of claim 96, wherein the ring contains two nitrogen atoms.

98. The kit of claim 97, wherein the drug comprises a DNA base or base derivative.

99. The kit of claim 35, wherein the drug comprises an alkyl chain and further comprising a unit selected from the group consisting of a carboxylate, a carboxylic acid, a phosphonate, a phosphoric acid, a primary amine and a secondary amine.

100. The kit of claim 99, wherein the drug is valproic acid sodium.

101. A kit comprising:
a composition having a high net polarization and comprising a structure selected from the group consisting of: 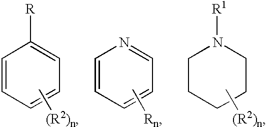
a five membered ring including at least one heteroatom, at least two rings bridged by at least one atom, at least two rings bonded directly to each other, a ring containing at least one carbonyl, and an alkyl chain;
wherein R and R² can be the same or different and each is a substituent of an organic group or chlorine, n is an integer greater than 0 and no more than 6 and each R can be the same or different and each R² can be the same or different; R¹ is a substituent of an organic group or hydrogen; any ring structure optionally including at least one carbon-carbon double bond; any ring structure optionally including at least one heteroatom selected from the group consisting of a carbonyl, nitrogen, oxygen and sulfur; each substituent being arranged so as to create polarity in the structure; and
instructions for use of the composition for treatment of disease.

102. A method comprising:
providing a composition having a high net polarization and comprising a structure selected from the group consisting of: 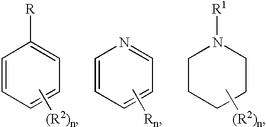
a five membered ring including at least one heteroatom, at least two rings bridged by at least one atom, at least two rings bonded directly to each other, a ring containing at least one carbonyl, and an alkyl chain;
wherein R and R² can be the same or different and each is a substituent of an organic group or chlorine, n is an integer greater than 0 and no more than 6 and each R can be the same or different and each R² can be the same or different; R¹ is a substituent of an organic group or hydrogen; any ring structure optionally including at least one carbon-carbon double bond; any ring structure optionally including at least one heteroatom selected from the group consisting of a carbonyl, nitrogen, oxygen and sulfur; each substituent being arranged so as to create polarity in the structure;
performing a combinatorial synthesis resulting in a plurality of compositions of the above structure; and
performing an assay involving a plurality of the compositions to determine their effectiveness in inhibiting neurodegenerative disease.

103. A kit, method, or composition as in any preceding claim, wherein the drug is selected from the group consisting of Atenolol, pindolol, histamine, methyl dihydrochloride, atenolol, 4-aminopyridine, physostigmine, and Tetracaine Hydrochloride.

104. A kit, method, or composition as in any preceding claim, wherein the drug is selected from the group consisting of sulpiride, uracil, 5-trifluoromethyl-5,6-dihydro, Atenolol, pindolol, BRL 37344 sodium, piperazine (2:1 ratio of 1-(4-hydroxy-3-methoxyphenyl)-1,2-ethanediol and diethyldiazene), cimetidine, methyl norepinephrine, oxotremorine methiodide, physostigmine, 4-aminopyridine, 1,3-dihydro-1-[2-hydroxy-5-(trifluoromethyl)phenyl]-5-(trifluoromethyl)-2H-benzimidazol-2-one), and erbstatin analog.

105. A method for treating a subject at risk of developing a disease or having a disease associated with fibril formation, comprising:
administering to the subject in need of such treatment an agent in an amount effective to inhibit fibril formation,
wherein the subject is otherwise free of symptoms calling for treatment with the agent.

106. The method of claim 105, wherein the agent is sulpiride or a homolog, analog, or derivative of sulpiride.

107. The method of claim 105, wherein the agent is uracil, 5-trifluoromethyl-5,6-dihydro or a homolog, analog, or derivative of uracil, 5-trifluoromethyl-5,6-dihydro.

108. The method of claim 105, wherein the agent is BRL 37344 sodium or a homolog, analog, or derivative of BRL 37344 sodium.

109. The method of claim 105, wherein the agent is S(−)−atenolol or a homolog, analog, or derivative of S(−)−atenolol.

110. The method of claim 105, wherein the agent is pindolol or a homolog, analog, or derivative of pindolol.

111. The method of claim 105, wherein the agent is MHPZ piperazine or a homolog, analog, or derivative of MHPZ piperazine.

112. The method of claim 105, wherein the agent is R(−)+atenolol or a homolog, analog, or derivative of R(−)+atenolol.

113. The method of claim 105, wherein the agent is cimetidine or a homolog, analog, or derivative of cimetidine.

114. The method of claim 105, wherein the agent is (−)-α-methyl norepinephrine or a homolog, analog, or derivative of (−)-α-methyl norepinephrine.

115. The method of claim 105, wherein the agent is oxotremorine methiodide or a homolog, analog, or derivative of oxotremorine methiodide.

116. The method of claim 105, wherein the agent is (−)-physostigmine or a homolog, analog, or derivative of (−)-physostigmine.

117. The method of claim 105, wherein the agent is 4-aminopyridine or a homolog, analog, or derivative of 4-aminopyridine.

118. The method of claim 105, wherein the agent is NS-1619 or a homolog, analog, or derivative of NS-1619.

119. The method of claim 105, wherein the agent is erbstatin analog or a homolog, analog, or derivative of erbstatin analog

120. The method of claim 105, wherein the agent is R(−)-α-methyl, dihydrochloride or a homolog, analog, or derivative of histamine, R(−)-α-methyl, dihydrochloride.

121. The method of claim 105, wherein the agent is tetracaine hydrochloride or a homolog, analog, or derivative of tetracaine hydrochloride.

122. The method of claim 105, wherein the agent is spironolactone or a homolog, analog, or derivative of spironolactone.

123. The method of claim 105, wherein the agent is uracil, 5-trifluoromethyl-5,6-dihydro or a homolog, analog, or derivative of uracil, 5-trifluoromethyl-5,6-dihydro.
